(19) United States
(12) Patent Application Publication
(10) Pub. No.: US 2003/0170642 A1
(43) Pub. Date: Sep. 11, 2003

(54) Identification of cellular targets for biologically active molecules

(75) Inventors: Jeremy S. Caldwell, Cardiff, CA (US); Sumit K. Chanda, La Jolla, CA (US); Nikunj V. Somia, Plymouth, MN (US); John B. Hogenesch, Encinitas, CA (US); Michael P. Cooke, Del Mar, CA (US); Pedro Aza-Blanc, San Diego, CA (US)

Correspondence Address: TIMOTHY L. SMITH, GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(73) Assignee: IRM, LLC

(21) Appl. No.: 10/097,554

(22) Filed: Mar. 12, 2002

Related U.S. Application Data
(60) Provisional application No. 60/275,266, filed on Mar. 12, 2001.

(51) Int. Cl. C12Q 1/68; G01N 33/53; G01N 33/567; C12N 5/06

(52) U.S. Cl. 435/6; 435/7.1; 435/7.21; 435/455; 435/320.1; 435/325

(57) ABSTRACT

A genetic screening methodology for rapid identification of candidate targets of any small molecule cellular effectors and other signals and modulators of cellular functions and pathways is provided. The effect of a small molecule or other signal on a cell is titrated by expressing within the cell cDNA that encodes a polypeptide that is the molecular target or that is responsible for directly or indirectly producing the molecular target.

### RELATED APPLICATIONS

Benefit of priority is claimed to U.S. provisional application Serial No. 60/275,266, filed Mar. 12, 2001, by Jeremy S. Caldwell, entitled, “IDENTIFICATION OF CELLULAR TARGETS FOR BIOLOGICALLY ACTIVE MOLECULES.”

This application is related to U.S. provisional application Serial No. 60/275,148, filed Mar. 12, 2001, by Jeremy S. Caldwell, entitled, “Chemical and Combinatorial Biology Strategies for High-Throughput Gene Functionalization;” U.S. provisional application Serial No. 60/274,979, filed Mar. 12, 2001, by Jeremy S. Caldwell, entitled, “Cellular Reporter Arrays;” and U.S. provisional application Serial No. 60/275,070, filed Mar. 12, 2001, by Andrew Su, John B. Hogenesch and Jeremy S. Caldwell, entitled, “Genomics-driven high speed cellular assay development.”

Where permitted, the subject matter of each of above-noted application are herein incorporated by reference in its entirety.

### FIELD OF INVENTION

Methods and materials for identifying cellular targets for the activity of biologically active molecules, such as small molecule effectors and other conditions that alter gene expression, are provided.

### BACKGROUND

Cell-based screening methods can identify small molecule effectors of complex signaling systems, but the identity of the molecular target is often unknown. The process, however, often is stymied because there are inadequate methods to determine the cellular targets of a small molecule effector found in a screen. Screening assays, thus, are generally black boxes. A cell is contacted or exposed to a perturbation, such as an effector molecule or condition, and an effect is observed. It, however, is not possible to identify with what a test compound or test condition is reacting or affecting in the cell. Many drug development campaigns are thwarted by the lack of target information. Without target information structure-activity relationship studies are impossible, and appropriate animal model tests and eventually phase I-III clinical trials can be hampered without target identification.

Hence there is a need to provide methods and products for performing cell-based assays and identifying the targets of any perturbations, including but not limited to, small effector molecules and other signals and conditions that affect cellular processes and activities. Therefore, it is an object herein to provide such methods and products.

### SUMMARY

Provided herein are methods and products for performing cell-based assays and identifying cellular pathways and targets of perturbations, including but not limited to, small effector molecules and other signals, and extra- and intracelluar changes, that affect cellular processes and activities. The cell-based screening methods and collections provided herein permit interrogation of complex cellular pathways and identification of critical components and perturbations, such as conditions, including effector molecules, that alter gene expression.

The methods permit identification of gene function in a genome or selected subportion thereof by modulating the level of message. The level of message can be modulated by increasing or decreasing the level of endogenous message or by adding exogenous nucleic acid, such as cDNA or RNA, including interfering RNA (siRNA), and antisense oligonucleotides to alter the total level of message in cells that report an output reflective of an activity.

The methods herein can be used to perform rational target selection by altering concentrations of components of pathways and observing the phenotypic results to permit identification of the rate limiting step(s) in a pathway. Typically the rate limiting step(s) is targeted. The methods also can be used to identify the target a characterized perturbation, such as an effector or condition.

Addressable collections of the reporter cells and cellular libraries and methods for production of the cells and collections thereof for use in the cell-base assay methods are provided. The cells are provided in addressable arrays, such as in or on positionally identifiable loci on a support, or linked to identifiable supports or labels. Each locus contains cells into which nucleic acid has been introduced. Each array includes a collection, such as a library of sets of cells. Different nucleic acid molecules are introduced into each set of cells. Since the arrays are addressable, the identify of the nucleic acid molecule introduced into cells at each locus is known or subsequently can be determined. Absent the nucleic acid molecules, the cells at each locus are identical. The resulting arrays serve as biosensors for assessing the effects of the added nucleic acid or of any perturbation or any signal or condition.

To produce the collections of cells with nucleic acids therein, each locus in a collection of cells is contacted with a different member of a nucleic acid molecule collection, such as a genomic library, a transcriptome library or nucleic acids encoding all molecules in a biological pathway or other collection under conditions whereby the nucleic acid is introduced into the cell. The resulting cells are used to assess different pathways, by looking for changes in gene expression by assessing resulting phenotypes and correlating them with the introduced nucleic acid molecule.

In high density formats, such as formats containing greater than 1500 loci, the reporter cells can be any cell as long as each locus has identical cells; such cells can be used to assay the effect(s) of any perturbation on the cells in very high density format; any selected output by the reporter cells can be monitored. In other embodiments, the cells are reporter cells that include a promoter linked to a reporter molecule or linked to other reporter function. The promoter is pre-selected to assess the effects of perturbations on a targeted pathway or set of genes. Methods for identifying promoters are known to those of skill in the art; other methods are described in copending U.S. application Ser. No. ______ (attorney dkt. no. 1311) filed on the same day herewith, claiming priority to provisional U.S. application Serial No. 60/275,070.

The methods provided herein include the steps of: 1) providing an addressable collection of reporter cells, such as in a multiwell plate in which two, generally three or more of the wells contain cells that produce an output in response to a perturbation, such as, but not limited to, expression of a reporter gene in response to exposure of the cell to an effector molecule or to an environmental change; 2) introducing nucleic acid molecules into the cells at each locus such that the different nucleic acid molecules are introduced into the cells at each locus for parallel screening, and 3) observing the effect on expression of a reporter gene or other output, such as trafficking, protein localization, proliferation and differentiation. Alteration of expression of a gene or derivative thereof that encodes a product or that is involved in a pathway the results in the changed phenotype indicates that such nucleic acid molecule encodes a product or blocks expression of a product in a pathway that results in the changed phenotype. Each nucleic acid that alters a phenotype can be annotated, such as by recording the information in a database.

In certain embodiments, the method is practiced by simultaneously, before or after introduction of the nucleic acid molecules, exposing the collection to a perturbation, such as contacting the cells with a modulator of an activity of interest, generally related to the gene from which the regulatory region linked to the reporter is derived, and then observing the effect on reporter expression or other output, such as trafficking, protein localization, proliferation, and differentiation.

Over-expression of a gene or derivative thereof that encodes a molecular target of a perturbation, such as an effector, in the cellular assay system treated with the perturbation can be detected as a change in the net effect of the perturbation on the readout. Candidate molecular targets of an perturbation are identified by screening gene expression collections in cells treated with the effector.

For example, a compound that inhibits an activity is identified. Sets of reporter cells that express a reporter gene whose expression is inhibited upon exposure to the compound are prepared or provided. Nucleic acid molecules such as members of a cDNA library are introduced into each, and the output is restoration of expression of the inhibited activity. Cells in which in which expression is restored are identified and, hence, the added nucleic acid is identified. The added cDNA encodes a product or is involved in the pathway targeted by the compound.

In another exemplary embodiment, for building a screen for a particular event or perturbation, a perturbation is replicated in cells in vitro and these cells are subsequently analyzed using addressable arrays, such as by adding nucleic acids from high density oligonucleotide arrays. Analysis of the effects on the cells in the resuling arrays yields a list of genes that change the response of the cells; comparison of this list to a database can further refine this list to genes that change specifically with respect to the introduced stimulus. Several of these specific responsive genes are identified, and their cognate promoters are identified in genomic DNA. These sequences are then specifically amplified using PCR upstream of the start methionine up to 10 kb. These candidate promoter regions are then cloned into a reporter vector, such as pGL3Basic (Promega). This reporter is subsequently tested in the presence or absence of the perturbation to validate that it accurately reflects the stimulus. Subsequently, the reporter and cDNAs or siRNAs can be co-transfected in the presence or absence of the perturbagen to identify: 1) genes that can mimic the perturbagen and therefore may be involved in the signaling, or 2) genes that complement the perturbagen and therefore may be involved in its signaling. These are the cellular genomic equivalents to a gain of function or modifier screens.

The output of the methods, such as fluorescence or other detectable signal, can be representative of gene expression, such as expression of a reporter gene, including, but are not limited to, a gene encoding a luciferase or fluorescent protein linked to a promoter in a pathway of interest, or a biochemical process or cellular activity, such as proliferation, differentiation, signal transduction and protein trafficking, which are assessed by standard methods known in the art. Thus, the method identifies, nucleic acid molecules whose introduction in a cell in the collections alters the output. The identified nucleic acid molecules or encoded products reverse, inhibit, enhance or otherwise alter the output, particularly in the presence of the perturbation.

In general, the methods observe the effects of the addition of nucleic acid molecules on each member of a collection of reporter cells by assessing phenotypic changes. The nucleic acid molecules can be added before, after or simultaneously with exposure of the cells in the addressable collection to a perturbation, such as a condition or change thereof or small molecule effector. The member cells of the addressable collection of reporter cells are substantially identical, but differ in the introduced nucleic acid, either or both in the sequence thereof or the amount thereof that is introduced into members of the collection. Cells that exhibit an altered response are identified. Since the collection is addressable, the identity of the added nucleic acid molecule is known or can be determined. Such nucleic acid molecule either is involved or encodes a product that is involved in a targeted pathway. The measurable effects of, for example, over-expressed molecular targets of effectors are enhanced by screening one gene per locus in an addressable collection. Parallel screening of one gene per locus increases the speed at such screens can be conducted and targets identified.

In particular, in cellulo competition methods in which the amount or level of a target molecule is changed are provided. The methods permit assessment of the effect(s) of a perturbation, such as, but not limited to, small molecule effectors, on cells, designated reporter cells. The effect(s) are titrated by modulating, such as increasing or inactivating, cellular levels of a molecular target or candidate target of the perturbation on cells that report an output reflective of an activity. Generally the level of the target is increased before, after or simultaneously with exposure or contact of the cells to a perturbation, such as a small molecule effector or a change in cellular environment. Modulating, such as increasing or inactivating, the level of target alters, typically decreases, the effect of the perturbation. Candidate targets that result in altered response to the perturbation, generally compared to a control, are identified. Hence, the method, which is performed on a plurality of reporter cells, permits parallel screening of a plurality of candidate cellular targets. In practicing embodiments of the methods, each of a plurality of nucleic acid molecules that encode potential targets or are potential targets are introduced into reporter cells. The resulting cells are exposed to the perturbation or perturbations of interest, either before, after or simultaneously with introduction of the nucleic acid molecules, and those potential targets that decrease or alter the effect of the perturbation are selected or identified as candidate targets. The nucleic acid molecules that are screened can be any collection of nucleic acid molecules, including libraries or subsets thereof. The reporter cells are cells that are designed produce a detectable output upon exposure to a selected perturbation, such as an condition or change thereof in the extracellular or intracellular environment or contact with a small effect molecule, a characterized or uncharacterized modulator of gene expression or any other such perturbagen of gene expression or gene product activity. The output can be detected or measured using any suitable device or means, such as standard plate readers, charge coupled devices (CCDs) and video monitors or even visually observed.

In an exemplary embodiment, transiently and stably transfected cells, such as the stably or transiently infected NFκB cells provided herein, are introduced into multiwell plates. Every cell-containing well is treated with a modulatory of activity of the pathway, and the response of the cells is monitored. Before, after or simultaneously with the contacting with the compound, each different member of a nucleic acid collection is introduced into the cells in each well. Differences in output in each well relative to the absence of an added nucleic acid molecule are detected. Any nucleic acid molecules that result in a change compared to the control well are candidates for the direct or indirect target of the compound.

In practicing the methods, perturbers, such as effectors and bio-active molecules and other conditions that alter gene expression or gene products are identified in any manner, including cell-based assays, in silico screening and other methods and combinations thereof. The effects of the perturbation can be measured or quantified. The effects of these perturbations on cells are modulated herein by altering the level of its target. By screening a plurality of cells to identify which different nucleic acid molecules, whose identity is known or can be determined, titrate effects of perturbations, such as small molecule effectors, potential targets for the perturbation are identified by screening for cells in which the effect of the perturbation is altered.

Thus, in certain embodiments, after adding the nucleic acid molecules, the cells are exposed to a perturbations, such as, but not limited to, contacting with a small molecule or subjecting the cells to a condition, and, detecting changes in an output relative to the absence of the nucleic acid molecule and, optionally in the absence of the perturbations, such as a signal. The nucleic acid molecule added to any that cells that exhibit a change from exposure to the perturation compared to a control therefor are candidates genes that express nucleic acid that is a direct or indirect target of the perturbation.

By screening a plurality of cells that express a different nucleic acid molecule in parallel, it is not necessary to deconvolute the identity of the gene because the identity of the nucleic acid added to each cell is known or can be known. Looking for things that reverse or inhibit or alter, enhance the change in the presence of the perturbation provides a way to do genetics on complex organisms, such as, animals, plants and microorgansims, including, but not limited to, mammals, including humans and rodents.

Methods for introducing the nucleic acid molecules into the cells are also provided.

Also provided are methods for transfection of nucleic acids into high density arrays of living cells, such as cells in multiwell plates at densities, 96, 384, 1536 wells or greater. Methods for parallel multi-well nucleic acid transfers, construction of cDNA expression matrices, and modifications of transfection procedures to facilitate protocol automation, cell transfection, and viral production in high-density plate formats are also provided.

Methods for introducing the nucleic acid molecules into cells, particularly into collections cells that are arrayed at or in discrete loci on a solid support, such as a microtiter plate, particularly high density plates (generally, although not necessarily, at least 96×n, where n is 4, 5, 6 . . . 100 or more or any other density, such as 500, 1500, 2000) are provided. The methods provided here are suitable for introduction of small amounts (sub-microgram) of nucleic acid molecules into cells at the high densities.

The method, which optionally is automated, is for transfection and transduction of cellular arrays with nucleic acid molecules of known identity, and hence can be used with the screening methods provided herein. An advantage of this technology is this increase in throughput over conventional transfections methods. Miniaturization and automation of the transfection/transduction procedure permits comprehensive studies of phenotypes and pathways at the level of the genome.

Each transfection is effected at a discrete addressable loci, such as in a positionally identifiable well on a high density microtiter palate. The resulting compartmentalized transfection permit whole cell lysis (i.e. for detecting a label such as a bioluminescence generating reaction such as one catalyzed by a luciferase, detection of secreted products, as well as viral production. Viral production permits transduction of cell that are not highly transfectable, as well as facilitate development of expanded timeline assays that require long-term retention of transduced genes.

The methods of transfection and transduction facilitate ultra high throughput cell-based functional analysis of nucleic acid molecules. Entire genomes can be functionally annotated for a given assay in one experiment. For example, the entire human transcriptome can be tested in fewer than about 100 plates. This platform can be also used for identification of genes and pathways disrupted by drug action or in phenotypic mutants through the gene complementation assays provided herein.

Furthermore, these methods permit use cDNA expression matrices to identify gene function. For example screens for “synthetic” or “dominant” lethal genes can be readily accomplished. This is in contrast to conventional cDNA library screens, which rely on selection of positive events, and subsequent deconvolution of cDNA identities. DNA matrix screen/assays require no deconvolution, since gene identity is ascertained by the address in the addressable array, such as by well location. This addressability obviates the requirement for “positive selection” events” and enables negative or lethal screens. Thus, these methods can be used to enhance any screen that relies on the introduction of nucleic acids into cells (i.e. mammalian two-hybrid, antisense, FRET, etc.), significantly expanding the scope of mammalian genetics.

All methods provided herein can be automated; hence automated cell-based assays for identifying cellular pathways and targets of perturbations, such as, but not limited to, small effector molecules and other signals, that affect cellular processes and activities. Systems for performing the assays and databases produced by the methods are also provided.

### DESCRIPTION OF DRAWINGS

FIG. 1A (top) shows Hek 293 NF-κB-luc clone time course/dose response. FIG. 1B (bottom) shows luciferase activity of Jurkat/NFκB cells induced with TNFα.

FIG. 2 shows the results of in cellulo competition experiments with (2A top) HEK293:NF-κB reporter cells (2A top) and Jurkat:NF-κB reporter cells (2B bottom).

FIG. 3 shows twelve compounds that were isolated by high density cell-based screening. Each compound was capable of blocking TNF-induced NF-κB activity as assessed by an NF-κB-dependent reporter cell assay. The name, compound structure and IC₅₀ value for each compound is shown.

FIG. 4 shows a scatter plot where the ID of the cDNA is on the x-axis and the activity of the over expressed cDNA in the HEK 293 NF-κB reporter cell line is on the y-axis.

FIG. 5 shows the effects of specific cDNA over expression on the effects of bioactive small molecules in a cellular reporter gene assay. These cells are HEK293 NF-κB-luciferase reporter cells. The stimulus or reagent introduced is shown on the x-axis. The y-axis shows the relative luciferase activity induced by each stimulus. The stars represent areas of interest.

### DETAILED DESCRIPTION

A. Definitions

Unless defined otherwise, all technical and scientific terms used herein have the same meaning as is commonly understood by one of skill in the art to which this invention belongs. All patents, patent applications, published patent applications and publications referred to herein are, unless noted otherwise, incorporated by reference in their entirety. In the event a definition in this section is not consistent with definitions elsewhere, the definition set forth in this section control. Reference to URLS and data available on the internet are exemplary only and provided to evidence the public availability of such information. Those of skill in the art can search for and identify equivalent information in electronic or hard copy formats using publicly and commercially available search tools.

As used herein, high-throughput screening (HTS) refers to processes that test a large number of samples, such as samples of test proteins or cells containing nucleic acids encoding the proteins of interest to identify structures of interest or the identify test compounds that interact with the variant proteins or cells containing them. HTS operations are amenable to automation and are typically computerized to handle sample preparation, assay procedures and the subsequent processing of large volumes of data.

As used herein, a perturbuation refers to any input that results in an altered cell response. Perturbations include any internal or external change in a cellular environment that results in an altered response compared to its absence. Thus, as used herein, a perturbation with reference to the cells refers to anything intra- or extra-cellular that alters gene expression or alters a cellular response. Perturbations include, but are not limited to, signals, such as those transduced by secondary messenger pathways, small effector molecules, including, for example, small organics, antisense, RNA and DNA, changes in intra or extracellular ion concentrations, such as changes in pH, Ca, Mg, Na and other ions, changes in temperature, pressure and concentration of any extracellular or intracellular component. Any such change or effector or condition is collectively referred to as a perturbation. The entity or condition that effects the perturbation is referred to as a “perturbagen.” As used herein, “targeted pathway” refers to a biochemical or cellular pathway that is under study. A pathway refers to a series of linked biochemical reactions or genes whose expression is linked.

As used herein, signals refer to transduced signals, such as those initiated by binding or removal or other interaction of a ligand with a cell surface receptor. Extracellular signals include an molecule or a change in the environment that is transduced intracellularly via cell surface proteins that interact, directly or indirectly, with the signal. An extracellular signal or effector molecule is any compound or substance that in some manner specifically alters the activity of a cell surface protein. Examples of such signals include, but are not limited to, molecules such as acetylcholine, growth factors, hormones and other mitogenic substances, such as phorbol mistric acetate (PMA), that bind to cell surface receptors and ion channels and modulate the activity of such receptors and channels. For example, antagonists are extracellular signals that block or decrease the activity of cell surface protein and agonists are examples of extracellular signals that potentiate, induce or otherwise enhance the activity of cell surface proteins.

As used herein, extracellular signals also include as yet unidentified substances that modulate the activity of a cell surface protein and thereby affecting intracellular functions and that are potential pharmacological agents that can be used to treat specific diseases by modulating the activity of specific cell surface receptors.

As used herein, “reporter” or “reporter moiety” refers to any moiety that allows for the detection of a molecule of interest, such as a protein expressed by a cell. Typical reporter moieties include, include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety can be expressed as a fusion protein with a protein of interest or under to the control of a promoter of interest. For the methods herein, reporters that are identifiable visually with a light detecting device are conveniently used. Patterns of light resulting from exposure of a collection of cells to a perturbation can be readily observed and saved as an image or a form derived therefrom. Pattern recognition software is optionally employed to identify resulting patterns.

As used herein, a reporter cell is a cell that can generate an output, a phenotype, in response to a perturbation. An exemplary reporter cell is one that expresses heterologous nucleic acid encoding a reporter moiety operably linked to a promoter and/or other regulatory region.

As used herein, identifying the target “for an effector” means finding an appropriate protein target to screen a perturbation, such as a small molecule modulator of that protein. In essence, the method provides a means for rational target selection by altering concentrations of components of pathways and observing the phenotypic results to permit identification of the rate limiting step(s) in a pathway. Typically the rate limiting step(s) is targeted.

As used herein, identifying the target “of an effector” or “of a perturbation” means having a perturbation, such as an effector or condition, that has a known effect and then finding the target that mediates the effect.

As used herein, chemiluminescence refers to a chemical reaction in which energy is specifically channeled to a molecule causing it to become electronically excited and subsequently to release a photon thereby emitting visible light. Temperature does not contribute to this channeled energy. Thus, chemiluminescence involves the direct conversion of chemical energy to light energy. Bioluminescence refers to the subset of chemiluminescence reactions that involve luciferins and luciferases (or the photoproteins). Bioluminescence does not herein include phosphorescence.

As used herein, bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives, for example, bacterial luciferin or firefly luciferase.

As used herein, luciferase refers to oxygenases that catalyze a light emitting reaction. For instance, bacterial luciferases catalyze the oxidation of flavin mononucleotide (FMN) and aliphatic aldehydes, which reaction produces light. Another class of luciferases, found among marine arthropods, catalyzes the oxidation of Cypridina (Vargula) luciferin, and another class of luciferases catalyzes the oxidation of Coleoptera luciferin.

Thus, luciferase refers to an enzyme or photoprotein that catalyzes a bioluminescent reaction (a reaction that produces bioluminescence). The luciferases, such as firefly and Renilla luciferases, that are enzymes which act catalytically and are unchanged during the bioluminescence generating reaction. The luciferase photoproteins, such as the aequorin and obelin photoproteins to which luciferin is non-covalently bound, are changed, such as by release of the luciferin, during bioluminescence generating reaction. The luciferase is a protein that occurs naturally in an organism or a variant or mutant thereof, such as a variant produced by mutagenesis that has one or more properties, such as thermal or pH stability, that differ from the naturally-occurring protein. Luciferases and modified mutant or variant forms thereof are well known.

Thus, reference, for example, to “Renilla luciferase” means an enzyme isolated from member of the genus Renilla or an equivalent molecule obtained from any other source, such as from another Anthozoa, or that has been prepared synthetically. The luciferases and luciferin and activators thereof are referred to as bioluminescence generating reagents or components.

As used herein, the component luciferases, luciferins, and other factors, such as O₂ Mg²⁺, Ca²⁺ are also referred to as bioluminescence generating reagents (or agents or components).

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases.

Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signal for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signal to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. It means a juxtaposition between two or more components so that the components are in a relationship permitting them to function in their intended manner. Thus, in the case of a regulatory region operatively linked to a reporter or any other gene sequence, or a reporter or any other gene sequence operatively linked to a regulatory region, expression of the gene/reporter is influenced or controlled (i.e., increased or decreased) by the regulatory region. For gene expression a DNA sequence and a regulatory sequence(s) are connected in such a way to control or permit gene expression when the appropriate molecular, e.g., transcriptional activator proteins, are bound to the regulatory sequence(s). Operative linkage of heterologous DNA to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a responder gene is a gene whose expression increases or decreases when a cell containing the gene or the gene is exposed to a perturbation, such as a small effector molecule, an extracellular signal, and a change in environment. Cells from an organism, or a tissue or an organ or other are exposed to a perturbation, and genes that have altered expression are identified. The genes that respond to the condition are referred to as responder genes. Exposure to different conditions will yield different sets of genes that are responders. In some embodiments, responders to a plurality of conditions are identified; in other embodiments, responders to a selected or particular condition, or from a particular cell type are selected. Subsets of the responder genes also can be identified. Once the responder genes are identified, regulatory regions, such as regions containing promoters, enhancers, transcription factor binding sites, translational regulatory regions, silencers and other such regulatory regions, are identified and isolated. The regulatory regions are each linked to nucleic acid encoding a reporter or to a nucleic acid reporter, and are introduced into cells. The resulting collection of cells is a collection of responder cells. Generally the collection is addressable (i.e., the identity of the regulatory region in each cell is known), such as by position on a substrate. Sub-collections of cells with different response patterns can be identified.

As used herein, robust responders refer to genes whose expression is increased or decreased substantially in response to a substance or stimulus. What is substantial depends upon the assay and reporting moiety. The precise increase, which can be empirically determined for each assay and/or collection of cells, should be sufficient to render the signals from reporters expressed from nucleic acid operatively linked to a robust responder regulatory region detectable under the conditions of the assay. Typically at least two-fold, generally at least a three-fold increase compared to other genes expressed when exposed to same perturbation and/or compared to the regulatory region in the absence of the perturbation or change thereof.

As used herein, receptor refers to a biologically active molecule that specifically binds to (or with) other molecules. The term “receptor protein” can be used to more specifically indicate the proteinaceous nature of a specific receptor. A receptor refers to a molecule that has an affinity for a given ligand. Receptors can be naturally-occurring or synthetic molecules. Receptors also can be referred to in the art as anti-ligands. As used herein, the receptor and anti-ligand are interchangeable. Receptors can be used in their unaltered state or as aggregates with other species. Receptors can be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic (ligand) selection;

b) antibodies: identification of a ligand-binding site on the antibody molecule that combines with the epitope of an antigen of interest can be investigated; determination of a sequence that mimics an antigenic epitope can lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant (see, e.g., U.S. Pat. No. 5,215,899);

e) hormone receptors: determination of the ligands that bind with high affinity to a receptor is useful in the development of hormone replacement therapies; for example, identification of ligands that bind to such receptors can lead to the development of drugs to control blood pressure; and

f) opiate receptors: determination of ligands that bind to the opiate receptors in the brain is useful in the development of less-addictive replacements for morphine and related drugs.

As used herein, antibody includes antibody fragments, such as Fab fragments, which are composed of a light chain and the variable region of a heavy chain.

As used herein, a ligand is a molecule that is specifically recognized by a particular receptor. Examples of ligands, include, but are not limited to, agonists and antagonists for cell membrane receptors, toxins and venoms, viral epitopes, hormones, such as steroids, hormone receptors, opiates, peptides, enzymes, enzyme substrates, cofactors, drugs, lectins, sugars, oligonucleotides, nucleic acids, oligosaccharides, proteins, and monoclonal antibodies.

As used herein, an anti-ligand is a molecule that has a known or unknown affinity for a given ligand and can be immobilized on a predefined region of the surface. Anti-ligands can be naturally-occurring or manmade molecules. Also, they can be employed in their unaltered state or as aggregates with other species. Anti-ligands can be reversibly attached, covalently or noncovalently, to a binding member, either directly or via a specific binding substance. By “reversibly attached” is meant that the binding of the anti-ligand (or specific binding member or ligand) is reversible and has, therefore, a substantially non-zero reverse, or unbinding, rate. Such reversible attachments can arise from noncovalent interactions, such as electrostatic forces, van der Waals forces, hydrophobic (i.e., entropic) forces and other forces. Furthermore, reversible attachments also can arise from certain, but not all covalent bonding reactions. Examples include, but are not limited to, attachment by the formation of hemiacetals, hemiketals, imines, acetals and ketals (see, e.g., Morrison et al. (1966) “Organic Chemistry”, 2nd ed., ch. 19). Examples of anti-ligands which can be employed in the methods and devices herein include, but are not limited to, cell membrane receptors, monoclonal antibodies and antisera reactive with specific antigenic determinants (such as on viruses, cells or other materials), hormones, drugs, oligonucleotides, peptides, peptide nucleic acids, enzymes, substrates, cofactors, lectins, sugars, oligosaccharides, cells, cellular membranes, and organelles.

As used herein, small amounts of nucleic acid (or protein) mean sub microgram amounts, including picogram and fentamole amounts.

As used herein, the term vector refers to a nucleic acid molecule capable of transporting another nucleic acid to which it has been linked, and include, but are not limited to, plasmids, cosmids and vectors of virus origin. Cloning vectors are typically used to genetically manipulate gene sequences while expression vectors are used to express the linked nucleic acid in a cell in vitro, ex vivo or in vivo. A vector that remains episomal contains at least an origin of replication for propagation in a cell; other vectors, such as retroviral vectors integrate into a host cell chromosome. One type of vector is an episome, i.e., a nucleic acid capable of extra-chromosomal replication.

Other vectors include are those capable of autonomous replication and/or expression of nucleic acids to which they are linked. Vectors capable of directing the expression of genes to which they are operatively linked are referred to herein as “expression vectors”. An “expression vector” therefore includes a gene regulatory region operatively linked to a sequence such as a reporter and can be propagated in cells. An “expression vector” can contain an origin of replication for propagation in a cell and includes a control element so that expression of a gene operatively linked thereto is influenced by the control element. Control elements include gene regulatory regions (e.g., promoters, transcription factor binding sites and enhancer elements) as set forth herein, that facilitate or direct or control transcription of an operatively linked sequence. “Plasmid” and “vector” are used interchangeably as the plasmid is the most commonly used form of vector. Other such other forms of expression vectors that serve equivalent functions and that become known in the art subsequently hereto. Vectors can include a selection marker.

As used herein, “selection marker” means a gene that allows selection of cells containing the gene. “Positive selection” means that only cells that contain the selection marker will survive upon exposure to the positive selection agent. For example, drug resistance is a common positive selection marker; cells containing a drug resistance gene will survive in culture medium containing the selection drug; whereas those which do not contain the resistance gene will die. Suitable drug resistance genes are neo, which confers resistance to G418, hygr, which confers resistance to hygromycin and puro, which confers resistance to puromycin. Other positive selection marker genes include reporter genes that allow identification by screening of cells. These genes include genes for fluorescent proteins (GFP), the lacZ gene (β-galactosidase), the alkaline phosphatase gene, and chlorampehnicol acetyl transferase. Vectors provided herein can contain negative selection markers.

As used herein, “negative selection” means that cells containing a negative selection marker are killed upon exposure to an appropriate negative selection agent. For example, cells that contain the herpes simplex virus-thymidine kinase (HSV-tk) gene are sensitive to the drug gancyclovir (GANC). Similarly, the gpt gene renders cells sensitive to 6-thioxanthine.

As used herein, self-inactivating (“SIN”) retroviral vectors are replication-deficient vectors that are created by deleting the promoter and enhancer sequences from the U3 region of the 3′ LTR (see, e.g., Yu et al. (1986) Proc, Natl. Acad. Sci. U.S.A. 83:3194-3198). Self-inactivating retrovirus have the 3′LTR and U3 regions removed so that upon recombination the LTR is gone A functional U3 region in the 5′ LTR permits expression of a recombinant viral genome in appropriate packaging lines. Upon expression of its genomic RNA and reverse transcription into cDNA, the U3 region of the 5′ LTR of the original provirus is deleted and replaced with defective U3 region of the 3′ LTR.

As a result, when a SIN vector integrates, then non-functional 3′ LTR replaces the functional 5′ LTR U3 region, rendering the virus incapable of expressing the full-length genomic transcript.

As used herein, “expression cassette” means a polynucleotide sequence containing a gene operatively linked to a control element (i.e. gene regulatory region) that can be transcribed and, if appropriate, translated. A gene regulatory region expression cassette includes a gene regulatory region of a responder, such as a robust responder, gene operatively linked to a sequence that encodes a reporter.

As used herein, a unidirection blocking sequence (utb) is a sequence of nucleotides that blocks expression of downstream nucleic acids (see, e.g., U.S. Pat. No. 5,583,022; vectors with such sequences available from Clontech). A utb avoids antisense effects created by two promoters that are on opposite strands.

As used herein, a scaffold attachment region (SAR) or a sequence that reduces or prevents nearby chromatin or adjacent sequences from influencing a promoter's control of the reporter gene. SARs insulate chromatin from nearby silencers and enhancers. In the constructs and vectors herein, a SAR is insulates the reporter construct from other genes. A SAR is not transcribed or translated, it is not a promoter or enhancer element. Its affect on gene expression is primarily position independent (see, U.S. Pat. No. 6,194,212, which describes the identification and use of SARs in retroviral vectors). Typically a SAR is at least 450 base pairs (bp) in length, generally from 600-1000 bp, such as about 800 bp. The SAR generally is AT-rich (i.e., more than 50%, typically more than 70% of the bases are adenine or thymine), and will generally include repeated 4-6 bp motifs, e.g., ATTA, ATTTA, ATTTTA, TAAT, TAAAT, TAAAAT, TAATA, and/or ATATTT, separated by spacer sequences, such as 3-20 bp, usually 8-12 bp, in length. The SAR can be from any eukaryote, such as a mammal, including a human. Suitably the SAR is the SAR for human IFN-β gene or a fragment thereof, such as a SAR derived from or corresponding to the 5′ SAR of human interferon beta (IFN-β) (see, Klehr et al. (1991) Biochemistry 30:1264-1270), including a fragment of at least 50 base pairs (bp) in length, typically from 600-1000 bp, such as about 800 bp, and being substantially homologous 115 to a corresponding portion of the 5′ SAR of a human IFN-β gene. By corresponding is meant having at least 80%, generally at least 90% or 95% homology therewith. An exemplary SAR is the 800 bp Eco-RI-HindIII (blunt end) fragment of the 5′ SAR element of IFN-β (see, Mielke et al.(1990) Biochemistry 29:7475-7485) or one that is at least 80%, 90%, and 95% homologous thereto.

As used herein, position independent means that functioning of a sequence does not require insertion into a specific site, but such sequence cannot be inserted such that other functioning sequences are destroyed.

As used herein, a transcriptome is a collection of transcripts from a genome, such a collection from a particular organ, cell, tissue, cell(s) or pathway. A transcriptome is a collection of RNA molecules (or cDNA produced therefrom) present in a cell, tissue or organ or other selected component of an animal or plant or other organism (see, e.g., Hoheisel et al. (1997) Trends Biotechnol. 15:465-469; Velculescu (1997) Cell 88:243-251 (1997).

As used herein, “a nucleic acid molecule represents a transcribed nucleic acid in a genome or transcriptome of a cell” means that the nucleic acid can modulate the level of the transcript in the cell. For example, the introduced nucleic acid molecule can be a cDNA that has a polynucleotide sequence that is at least substantially identical to all or part of that of the endogenous transcribed nucleic acid such that, when transcribed, the introduced nucleic acid molecule results in an increase in the copy number of transcripts corresponding to the endogenous transcribed nucleic acid. Alternatively, the introduced nucleic acid molecule can decrease the copy number of transcripts that correspond to the endogenous transcribed nucleic acid. For example, the introduced nucleic acid can be, or can be transcribed to yield, an antisense RNA, an RNAi or an siRNA molecule that has a sequence that is at least substantially identical to at least a portion of the endogenous transcribed nucleic acid or a transcript of such endogenous nucleic acid.

### Solid Supports, Chips, Arrays and Collection

As used herein, a collection contains two, generally three, or more elements.

As used herein, an array refers to a collection of elements, such as nucleic acid molecules, containing three or more members; arrays can be in solid phase or liquid phase. An addressable array or collection is one in which each member of the collection is identifiable typically by position on a solid phase support or by virtue of an identifiable or detectable label, such as by color, fluorescence, electronic signal (i.e. RF, microwave or other frequency that does not substantially alter the interaction of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. The collection can be in the liquid phase if other discrete identifiers, such as chemical, electronic, colored, fluorescent or other tags are included.

As used herein, a substrate (also referred to as a matrix support, a matrix, an insoluble support, a support or a solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be a be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, are also contemplated. The “beads” can include additional components, such as magnetic or paramagnetic particles (see, e.g., Dyna beads (Dynal, Oslo, Norway)) for separation using magnets, as long as the additional components do not interfere with the methods and analyses herein. For the collections of cells, the substrate should be selected so that it is addressable (i.e., identifiable) and such that the cells are linked, absorbed, adsorbed or otherwise retained thereon.

As used herein, a substrate (also referred to as a matrix support, a matrix, an insoluble support, a support or a solid support) refers to any solid or semisolid or insoluble support to which a molecule of interest, typically a biological molecule, organic molecule or biospecific ligand is linked or contacted. A substrate or support refers to any insoluble material or matrix that is used either directly or following suitable derivatization, as a solid support for chemical synthesis, assays and other such processes. Substrates contemplated herein include, for example, silicon substrates or siliconized substrates that are optionally derivatized on the surface intended for linkage of anti-ligands and ligands and other macromolecules. Other substrates are those on which cells adhere.

Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications.

Thus, a substrate, support or matrix refers to any solid or semisolid or insoluble support on which the molecule of interest, typically a biological molecule, macromolecule, organic molecule or biospecific ligand or cell is linked or contacted. Typically a matrix is a substrate material having a rigid or semi-rigid surface. In many embodiments, at least one surface of the substrate is substantially flat or is a well, although in some embodiments it can be desirable to physically separate synthesis regions for different polymers with, for example, wells, raised regions, etched trenches, or other such topology. Matrix materials include any materials that are used as affinity matrices or supports for chemical and biological molecule syntheses and analyses, such as, but are not limited to: polystyrene, polycarbonate, polypropylene, nylon, glass, dextran, chitin, sand, pumice, polytetrafluoroethylene, agarose, polysaccharides, dendrimers, buckyballs, polyacrylamide, Kieselguhr-polyacrlamide non-covalent composite, polystyrene-polyacrylamide covalent composite, polystyrene-PEG (polyethyleneglycol) composite, silicon, rubber, and other materials used as supports for solid phase syntheses, affinity separations and purifications, hybridization reactions, immunoassays and other such applications.

The substrate, support or matrix herein can be particulate or can be a be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, are also contemplated. The “beads” can include additional components, such as magnetic or paramagnetic particles (see, e.g., Dyna beads (Dynal, Oslo, Norway)) for separation using magnets, as long as the additional components do not interfere with the methods and analyses herein. For the collections of cells, the substrate should be selected so that it is addressable (i.e., identifiable) and such that the cells are linked, absorbed, adsorboed or otherwise retained thereon.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be order of cubic microns. Such particles are collectively called “beads.”

As used herein, high density arrays refer to arrays that contain 384 or more, including 1536 or more or any multiple of 96 or other selected base, loci per support, which is typically about the size of a standard 96 well microtiter plate. Each such array is typically, although not necessarily, standardized to be the size of a 96 well microtiter plate. It is understood that other numbers of loci, such as 10, 100, 200, 300, 400, 500, 10ⁿ, wherein n is any number from 0 and up to 10 or more. Ninety-six is merely an exemplary number. For addressable collections that are homogeneous (i.e. not affixed to a solid support), the numbers of members are generally greater. Such collections can be labeled chemically, electronically (such as with radio-frequency, microwave or other detectable electromagnetic frequency that does not substantially interfere with a selected assay or biological interaction).

As used herein, the attachment layer refers the surface of the chip device to which molecules are linked. A chip can be a silicon semiconductor device, which is coated on a least a portion of the surface to render it suitable for linking molecules and inert to any reactions to which the device is exposed. Molecules are linked either directly or indirectly to the surface, linkage can be effected by absorption or adsorption, through covalent bonds, ionic interactions or any other interaction. Where necessary the attachment layer is adapted, such as by derivatization for linking the molecules.

As used herein, a gene chip, also called a genome chip and a microarray, refers to high density oligonucleotide-based arrays. Such chips typically refer to arrays of oligonucleotides for designed monitoring an entire genome, but can be designed to monitor a subset thereof. Gene chips contain arrayed of polynucleotide chains (oligonucleotides of DNA or RNA or nucleic acid analogs or combinations thereof) that are single-stranded, or at least partially or completely single-stranded prior to hybridization. The oligonucleotides are designed to specifically and generally uniquely hybridize to particular genes in a population, whereby by virtue of formation of a hybrid the presence of a gene in a population can be identified. Gene chips are commercially available or can be prepared. Exemplary microarrays include the Affymetrix GeneChip® arrays. Such arrays are typically fabricated by high speed robotics on glass, nylon or other suitable substrate, and include a plurality of probes (oligonucleotides) of known identity defined by their address in (or on) the array (an addressable locus). The oligonucleotides are used to determine complementary binding and to thereby provide parallel gene expression and gene discovery in a sample containing target nucleic acid molecules. Thus, as used herein, a gene chip refers to an addressable array, typically a two-dimensional array, that includes plurality of oligonucleotides associate with addressable loci “addresses”, such as on a surface of a microtiter plate or other solid support.

As used herein, a plurality of genes includes at least two, five, 10, 25, 50, 100, 250, 500, 1000, 2,500, 5,000, 10,000, 100,000, 1,000,000 or more genes. A plurality of genes can include complete or partial genomes of an organism or even a plurality thereof. Selecting the organism type determines the genome from among which the gene regulatory regions are selected. Exemplary organisms for gene screening include animals, such as mammals, including human and rodent, such as mouse, insects, yeast, bacteria, parasites, and plants.

As used herein, transcriptome is a collection of transcripts from a genome, such as a collection from a particular organ, cell, tissue, cell(s) exposed to a perturbation. A transcriptome is a collection of RNA molecules (or cDNA produced therefrom) present in a cell, tissue or organ or other selected component of an animal or plant or other organism (see, e.g., Hoheisel et al. (1997) Trends Biotechnol. 15:465-469).

Recombinases

As used herein, recognition sequences are particular sequences of nucleotides that a protein, DNA, or RNA molecule, such as, but are not limited to, a restriction endonuclease, a modification methylase and a recombinase) recognizes and binds. For example, a recognition sequence for Cre recombinase (see, e.g., SEQ ID 4 is a 34 base pair sequence containing two 13 base pair inverted repeats (serving as the recombinase binding sites) flanking an 8 base pair core and designated loxP (see, e.g., Sauer (1994) Current Opinion in Biotechnology 5:521-527).

As used herein, a recombinase is an enzyme that catalyzes the exchange of DNA segments at specific recombination sites. An integrase herein refers to a recombinase that is a member of the lambda (A) integrase family.

As used herein, recombination proteins include excisive proteins, integrative proteins, enzymes, co-factors and associated proteins that are involved in recombination reactions using one or more recombination sites (see, Landy (1993) Current Opinion in Biotechnology 3:699-707).

As used herein the expression “lox site” means a sequence of nucleotides at which the gene product of the cre gene, referred to herein as Cre, can catalyze a site-specific recombination. A LoxP site is a 34 base pair nucleotide sequence from bacteriophage P1 (see, e.g., Hoess et al. (1982) Proc. Natl. Acad. Sci. U.S.A. 79:3398-3402). The LoxP site contains two 13 base pair inverted repeats separated by an 8 base pair spacer region as follows: (SEQ ID NO. 4):

ATAACTTCGTATA ATGTATGC TATACGAAGTTAT

E. coli DH5Δ/ac and yeast strain BSY23 were transformed with plasmid pBS44 carrying two loxP sites connected with a LEU2 gene are available from the American Type Culture Collection (ATCC) under accession numbers ATCC 53254 and ATCC 20773, respectively. The lox sites can be isolated from plasmid pBS44 with restriction enzymes Eco RI and Sal I, or Xho I and Bam I. In addition, a preselected DNA segment can be inserted into pBS44 at either the Sal I or Bam I restriction enzyme sites. Other lox sites include, but are not limited to, LoxB, LoxL, LoxC2 and LoxR sites, which are nucleotide sequences isolated from E. coli (see, e.g., Hoess et al. (1982) Proc. Natl. Acad. Sci. U.S.A. 79:3398). Lox sites also can be produced by a variety of synthetic techniques (see, e.g., Ito et al. (1982) Nuc. Acid Res. 10:1755 and Ogilvie et al. (1981) Science 270:270.

As used herein, the expression “cre gene” means a sequence of nucleotides that encodes a gene product that effects site-specific recombination of DNA in eukaryotic cells at lox sites. One cre gene can be isolated from bacteriophage P1 (see, e.g., Abremski et al. (1983) Cell 32:1301-1311). E. coli DH1 and yeast strain BSY90 transformed with plasmid pBS39 carrying a cre gene isolated from bacteriophage P1 and a GAL1 regulatory nucleotide sequence are available from the American Type Culture Collection (ATCC) under accession numbers ATCC 53255 and ATCC 20772, respectively. The cre gene can be isolated from plasmid pBS39 with restriction enzymes Xho I and Sal I.

As used herein, site specific recombination refers site specific recombination that is effected between two specific sites on a single nucleic acid molecule or between two different molecules that requires the presence of an exogenous protein, such as an integrase or recombinase.

For example, Cre-lox site-specific recombination includes the following three events:

a. deletion of a pre-selected DNA segment flanked by lox sites;

b. inversion of the nucleotide sequence of a pre-selected DNA segment flanked by lox sites; and

c. reciprocal exchange of DNA segments proximate to lox sites located on different DNA molecules.

This reciprocal exchange of DNA segments can result in an integration event if one or both of the DNA molecules are circular. DNA segment refers to a linear fragment of single- or double-stranded deoxyribonucleic acid (DNA), which can be derived from any source. Since the lox site is an asymmetrical nucleotide sequence, two lox sites on the same DNA molecule can have the same or opposite orientations with respect to each other. Recombination between lox sites in the same orientation result in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the gene product of the cre gene. Thus, the Cre-lox system has can be used to specifically excise, delete or insert DNA. The precise event is controlled by the orientation of lox DNA sequences, in cis the lox sequences direct the Cre recombinase to either delete (lox sequences in direct orientation) or invert (lox sequences in inverted orientation) DNA flanked by the sequences, while in trans the lox sequences can direct a homologous recombination event resulting in the insertion of a recombinant DNA.

General Definitions

As used herein, biological and pharmacological activity includes any activity of a biological pharmaceutical agent and includes, but is not limited to, biological efficiency, transduction efficiency, gene/transgene expression, differential gene expression and induction activity, titer, progeny productivity, toxicity, cytotoxicity, immunogenicity, cell proliferation and/or differentiation activity, anti-viral activity, morphogenetic activity, teratogenetic activity, pathogenetic activity, therapeutic activity, tumor suppressor activity, ontogenetic activity, oncogenetic activity, enzymatic activity, pharmacological activity, cell/tissue tropism and delivery.

As used herein, phenotype refers to the physical or other manifestation of a genotype (a sequence of a gene). In the methods herein, phenotypes that result from alteration of a genotype are assessed.

As used herein, “effect the phenotype” means cause a phenotype by producing it, or influencing it, or otherwise alter gene expression that is directly or indirectly responsible for the the phenotype

As used herein, the amino acids, which occur in the various amino acid sequences appearing herein, are identified according to their known, three-letter or one-letter abbreviations (see, Table 1). The nucleotides, which occur in the various nucleic acid fragments, are designated with the standard single-letter designations used routinely in the art.

As used herein, “loss-of-function” sequence, as it refers to the effect of a polynucleotide such as antisense nucleic acid, siRNA and cDNA, refers to those sequences which, when expressed in a host cell, inhibit expression of a gene or otherwise render the gene product thereof to have substantially reduced activity, or preferably no activity relative to one or more functions of the corresponding wild-type gene product.

As used herein, amino acid residue refers to an amino acid formed upon chemical digestion (hydrolysis) of a polypeptide at its peptide linkages. The amino acid residues described herein are presumed to be in the “L” isomeric form. Residues in the “D” isomeric form, which are so-designated, can be substituted for any L-amino acid residue, as long as the desired functional property is retained by the polypeptide; such residues. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in the following Table:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. (1987) Molecular Biology of the Gene, 4th Edition, The Benjamin/Cummings Pub. co., p.224).

Such substitutions are preferably made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions.

As used herein, a biopolymer includes, but is not limited to, nucleic acid, proteins, polysaccharides, lipids and other macromolecules. Nucleic acids include DNA, RNA, and fragments thereof. Nucleic acids can be isolated or derived from genomic DNA, RNA, mitochondrial nucleic acid, chloroplast nucleic acid and other organelles with separate genetic material or can be prepared synthetically.

As used herein, nucleic acids include DNA, RNA and analogs thereof, including protein nucleic acids (PNA) and mixture thereof. Nucleic acids can be single or double stranded. When referring to probes or primers, optionally labeled with a detectable label, such as a fluorescent or radiolabel, single-stranded molecules are contemplated. Such molecules are typically of a length such that they are statistically unique of low copy number (typically less than 5, preferably less than 3) for probing or priming a library. Generally a probe or primer contains at least 14, 16 or 30 contiguous of sequence complementary to or identical a gene of interest. Probes and primers can be 10, 14, 16, 20, 30, 50, 100 or more nucleic acid bases long.

As used herein, “oligonucleotide,” “polynucleotide” and “nucleic acid” include linear oligomers of natural or modified monomers or linkages, including deoxyribonucleosides, ribonucleotides, α-anomeric forms thereof capable of specifically binding to a target gene by way of a regular pattern of monomer-to-monomer interactions, such as Watson-Crick type of base pairing, base stacking, Hoogsteen or reverse Hoogsteen types of base pairing. Monomers are typically linked by phosphodiester bonds or analogs thereof to form the oligonucleotides. Whenever an oligonucleotide is represented by a sequence of letters, such as “ATGCCTG,” it is understood that the nucleotides are in a 5′->3′ order from left to right.

Typically oligonucleotides for hybridization include the four natural nucleotides; however, they also can include non-natural nucleotide analogs, derivatized forms or mimetics. Analogs of phosphodiester linkages include phosphorothioate, phosphorodithioate, phosphorandilidate, phosphoramidate, for example. A particular example of a mimetic is protein nucleic acid (see, e.g., Egholm et al. (1993) Nature 365:566; see also U.S. Pat. No. 5,539,083).

As used herein, labels include any composition or moiety that can be attached to or incorporated into nucleic acid that is detectable by spectroscopic, photochemical, biochemical, immunochemical, electrical, optical or chemical means. Exemplary labels include, but are not limited to, biotin for staining with labeled streptavidin conjugate, magnetic beads (e.g., DynabeadsTM), fluorescent dyes (e.g., 6-FAM, HEX, TET, TAMRA, ROX, JOE, 5-FAM, R110, fluorescein, texas red, rhodamine, lissamine, phycoerythrin (Perkin Elmer Cetus), Cy2, Cy3, Cy3.5, Cy5, Cy5.5, Cy7, FluorX (Amersham), radiolabels, enzymes (e.g., horse radish peroxidase, alkaline phosphatase and others used in ELISA), and colorimetric labels such as colloidal gold or colored glass or plastic (e.g., polystyrene, polypropylene, latex and other supports) beads, a fluorophore, a radioisotope or a chemiluminescent moiety.

As used herein, “mistmatch control” means a sequence that is not perfectly complementary to a particular oligonucleotide. The mismatch can include one or more mismatched bases. The mismatch(s) can be located at or near the center of the probe such that the mismatch is most likely to destabilize the duplex with the target sequence under hybridization conditions, but can be located anywhere, for example, a terminal mismatch. The mismatch control typically has a corresponding test probe that is perfectly complementary to the same particular target sequence. Mismatches are selected such that under appropriate hybridization conditions the test or control oligonucleotide hybridizes with its target sequence, but the mismatch oligonucleotide does not. Mismatch oligonucleotides therefore indicate whether hybridization is specific or not. For example, if the target gene is present the perfect match oligonucleotide should be consistently brighter than the mismatch oligonucleotide.

As used herein, nucleic acid derived from an RNA means that the RNA has ultimately served as a template. Thus, a cDNA reverse transcribed from an mRNA, an RNA transcribed from that cDNA, a DNA amplified from the cDNA, an RNA transcribed from the amplified DNA are derived from an RNA and using such derived products to determine changes in gene expression are included. Thus, suitable nucleic acids include, but are not limited to, mRNA transcripts of the gene or genes, cDNA reverse transcribed from the mRNA, cRNA transcribed from the cDNA, DNA amplified from the genes and RNA transcribed from amplified DNA.

As used herein, amplifying refers to means for increasing the amount of a biopolymer, especially nucleic acids. Based on the 5′ and 3′ primers that are chosen, amplification also serves to restrict and define the region of the genome which is subject to analysis. Amplification can be by any means known to those skilled in the art, including use of the polymerase chain reaction (PCR) and other amplification protocols, such as ligase chain reaction, RNA replication, such as the autocatalytic replication catalyzed by, for example, Qβ replicase. Amplification is done quantitatively when the frequency of a polymorphism is determined.

As used herein, small interfering RNA (siRNA) refers to dsRNA that specifically degrades endogenous message encoded a targeted protein. siRNA is prepared by identifying a target sequence of nucleotides in DNA, such as about 20-30, is selected to be identical and complementary to a target sequence.

As used herein, cleaving refers to non-specific and specific fragmentation of a biopolymer.

As used herein, by homologous means about greater than 25% nucleic acid or amino acid sequence identity, generally 25% 40%, 60%, 80%, 90% or 95%. The intended percentage will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

As used herein, a nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function. Ppolypeptide homologs would be polypeptides that could be encoded substantially identical (i.e., 80%, 90%, 95% identifical) sequences of nucleotides.

The terms “homology” and “identity” are often used interchangeably. In this regard, percent homology or identity can be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program can include: (1) a unitary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are, for example, at least 80%, 85%, 90%, 95%, 96%, 97%, 98% or 99%, “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database can be used to determine identity. In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux et al. (1984) Nucleic Acids Research 12(1):387), BLASTP, BLASTN, FASTA (Atschul, S.F., et al., J Molec Biol 215:403 (1990)), and CLUSTALW. For sequences displaying a relatively high degree of homology, alignment can be effected manually by simpling lining up the sequences by eye and matching the conserved portions.

Therefore, as used herein, the term “identity” represents a comparison between a test and a reference polypeptide or polynucleotide. For example, a test polypeptide can be defined as any polypeptide that is 90% or more identical to a reference polypeptide. Alignment can be performed with any program for such purpose using default gap parameters and penalties or those selected by the user. For example, a program called CLUSTALW program can be employed with parameters set as follows: scoring matrix BLOSUM, gap open 10, gap extend 0.1, gap distance 40% and transitions/transversions 0.5; specific residue penalties for hydrophobic amino acids (DEGKNPQRS), distance between gaps for which the penalties are augmented was 8, and gaps of extremities penalized less than internal gaps.

As used herein, substantially identical to a product means sufficiently similar so that the property of interest is sufficiently unchanged so that the substantially identical product can be used in place of the product.

As used herein, a “corresponding” position on a protein (or nucleic acid molecule) refers to an amino acid position (or nucleotide base position) based upon alignment to maximize sequence identity between or among related proteins(or nucleic acid molecules).

As used herein, the term at least “90% identical to” refers to percent identities from 90 to 100% relative to reference polypeptides or nucleic acid moleucles. Identity at a level of 90% or more is indicative of the fact that, assuming for exemplification purposes a test and reference polypeptide (or polynucleotide) length of 100 amino acids are compared. No more than 10% (i.e., 10 out of 100) amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions.

As used herein, it is also understood that the terms substantially identical or similar varies with the context as understood by those skilled in the relevant art.

As used herein, “hybridization” refers to the binding between complementary nucleic acids. “Selective hybridization” refers to hybridization that distinguishes related sequences from unrelated sequences. Hybridization conditions will be such that an oligonucleotide will hybridize to its target nucleic acid, but not significantly to non-target sequences. As is understood by those skilled in the art, the T_{M} (melting temperature) refers to the temperature at which binding between complementary sequences is no longer stable. For two nucleic acid sequences to bind, the temperature of a hybridization reaction must be less than the calculated T_{M} for the sequences. The T_{M} is influenced by the amount of sequence complementarity, length, composition (%GC), type of nucleic acid (RNA vs. DNA), and the amount of salt, detergent and other components in the reaction (e.g., formamide). For example, longer hybridizing sequences are stable at higher temperatures. Duplex stability between RNA, DNA and mixtures thereof is generally in the order of RNA:RNA>RNA:DNA>DNA:DNA. All of these factors are considered in establishing appropriate hybridization conditions (see, e.g., the hybridization techniques and formula for calculating TM described in Sambrook et al. (1989) Molecular Cloning: A Laboratory Manual (2nd Ed.), Vol. 1-3, Cold Spring Harbor Laboratory, Cold Spring Harbor, N.Y.). Generally, stringent conditions are selected to be about 5° C. lower than the melting point (Tm) for the specific sequence at a defined ionic strength and pH.

Typically, wash conditions are adjusted so as to attain the desired degree of hybridization stringency. Thus, hybridization stringency can be determined empirically, for example, by washing under particular conditions, e.g., at low stringency conditions or high stringency conditions. Optimal conditions for selective hybridization will vary depending on the particular hybridization reaction involved. An exemplary gene chip hybridization is described in Example 1.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C.

Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al, vol. 3, p. B.13, see, also, numerous catalogs that describe commonly used laboratory solutions). It is understood that equivalent stringencies can be achieved using alternative buffers, salts and temperatures.

As used herein equivalent, when referring to two sequences of nucleic acids means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 2) that do not substantially alter the activity or function of the protein or peptide. When “equivalent” refers to a property, the property does not need to be present to the same extent (e.g., peptides can exhibit different rates of the same type of enzymatic activity), but the activities are preferably substantially the same. “Complementary,” when referring to two nucleotide sequences, means that the two sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein, heterologous or foreign nucleic acid, such as DNA and RNA, are used interchangeably and refer to DNA or RNA that does not occur naturally as part of the genome in which it is present or which is found in a location or locations in the genome that differ from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by a cell in which it is expressed. Any DNA or RNA that one of skill in the art would recognize or consider as heterologous or foreign to the cell in which it is expressed is herein encompassed by heterologous DNA. Heterologous DNA and RNA also can encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and DNA that encodes other types of proteins, such as antibodies.

Hence, herein heterologous DNA or foreign DNA, includes a DNA molecule not present in the exact orientation and position as the counterpart DNA molecule found in the genome. It also can refer to a DNA molecule from another organism or species (i.e., exogenous).

As used herein, a sequence complementary to at least a portion of an RNA, with reference to antisense oligonucleotides, means a sequence having sufficient complementarily to be able to hybridize with the RNA, preferably under moderate or high stringency conditions, forming a stable duplex. The ability to hybridize depends on the degree of complementarily and the length of the antisense nucleic acid. The longer the hybridizing nucleic acid, the more base mismatches it can contain and still form a stable duplex (or triplex, as the case can be). One skilled in the art can ascertain a tolerable degree of mismatch by use of standard procedures to determine the melting point of the hybridized complex.

As used herein, isolated with reference to a nucleic acid molecule or polypeptide or other biomolecule means that the nucleic acid or polypeptide has separated from the genetic environment from which the polypeptide or nucleic acid were obtained. It also can mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compounds can be substantially purified by the one-step method described in Smith and Johnson, Gene 67:31-40 (1988). The terms isolated and purified are sometimes used interchangeably.

Thus, by “isolated” is meant that the nucleic is free of the coding sequences of those genes that, in the naturally-occurring genome of the organism (if any) immediately flank the gene encoding the nucleic acid of interest. Isolated DNA can be single-stranded or double-stranded, and can be genomic DNA, cDNA, recombinant hybrid DNA, or synthetic DNA. It can be identical to a native DNA sequence, or can differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures can include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, referred to as a single nucleotide polymorphism (SNP), the identity of which differs in different alleles. A polymorphic region also can be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is the to be homozygous for the gene or allele. When a subject has two different alleles of a gene, the subject is the to be heterozygous for the gene. Alleles of a specific gene can differ from each other in a single nucleotide, or several nucleotides, and can include substitutions, deletions, and insertions of nucleotides. An allele of a gene also can be a form of a gene containing a mutation.

As used herein, the term “gene” or “recombinant gene” refers to a nucleic acid molecule containing an open reading frame and including at least one exon and (optionally) an intron sequence. A gene can be either RNA or DNA. Genes can include regions preceding and following the coding region (leader and trailer).

As used herein, “intron” refers to a DNA sequence present in a given gene which is spliced out during mRNA maturation.

As used herein, “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, production by recombinant means by using recombinant DNA methods means the use of the known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds. It can be a solution, a suspension, liquid, powder, a paste, aqueous, non-aqueous or any combination thereof.

As used herein, a combination refers to any association between two or more items. A combination can be packaged as a kit As used herein, “packaging material” refers to a physical structure housing the components (e.g., one or more regulatory regions, reporter constructs containing the regulatory regions or cells into which the reporter constructs have been introduced) of the kit. The packaging material can maintain the components sterilely, and can be made of material and containers commonly used for such purposes (e.g., paper, corrugated fiber, glass, plastic, foil, ampules, vials, tubes and others). The label or packaging insert can include appropriate written instructions, for example, practicing a method provided herein.

As used herein, the “database” means a collection of information, such as information (i.e., sequences) representative of two or more regulatory regions. Databases are typically present on computer readable medium so that they can be accessed and analyzed.

As used herein, the singular forms “a”, “and,” and “the” include plural referents unless the context clearly indicates otherwise. Thus, for example, reference to “a gene regulatory region” includes a plurality of such regulatory regions and reference to “a responder cell” includes reference to one or more such responder cells (e.g., a collection or library of responder cells), and so forth.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Cell-Based Screening Processes

Cell-based screening processes can identify bioactive molecules and other effectors, such as small molecules, that modulate complex signaling systems, but the identity of the molecular target is often unknown. Methods provided herein permit the use of effectors of complex pathways, to rapidly identify candidate targets of any cellular effector. In practicing methods provided herein, the effect of an perturbation, such as a small molecule, on cells is titrated by changing cellular levels of its molecular target, such as polypeptides, including but are not limited to, receptors and enzymes, nucleic acid molecules, lipids, carbohydrates, other small molecules such as co-factor. As provided herein, the effect of a small molecule with a known target is titrated by over-expression of its molecular target. Hence, the process involves in cellulo competition. By screening a plurality of cells, each titrated with a different nucleic acid molecule, with an effector targets of the effector are identified. The different nucleic acid molecules constitute a collection of molecules whose identity is known or whose identity is know or can be determined. The resulting genetic screening methodologies are used to identify molecular targets of any cellular effector.

The observed effects can be modulated by altering levels of a target(s). The observed output of the cellular assay depends on the mode of action, such as agonist, antagonist, inverse agonist and other modes action, of the effector. For example:

a) inhibition of a cellular readout by treatment with a small molecule can be diminished by introducing to that cell higher levels of its molecular target;

b) inhibition of a cellular readout by treatment with a small molecule can be potentiated by introducing to that cell levels of a mutant form of its molecular target;

c) activation of a cellular readout by treatment with a small molecule can be potentiated by introducing to that cell higher levels of its molecular target; and

d) activation of a cellular readout by treatment with a small molecule can be diminished by introducing to that cell levels of a mutant form of its molecular target.

Over-expression of a gene or derivative of a gene encoding the molecular target of a given bioactive small molecule in a cellular assay system treated with the small molecule as a change in the net effect of the small molecule on the cell readout is detected. Candidate molecular targets of the molecules or other signals can be identified by screening gene expression libraries in cells treated with a small molecule of interest. The measurable effects of over-expressed molecular targets of the molecules or other signals is greatly enhanced by screening one gene per test or well. Parallel screening of one gene per well significantly increases the speed at which such small molecule complementation screens can be performed and targets are identified. The parallel screening process routinely used to screen small molecule libraries can be applied to gene expression libraries to enhance this process.

In practicing the methods, a cDNA or other library from a selected target genome or a portion thereof, such as the human genome, is sampled in parallel by introducing each cDNA molecule, or mixtures or pools thereof, into cells that contain reporter constructs in addressable collections to quickly find subsets that modulate observed effect of exposure to a perturbation, such as a compound. One or a plurality of the subsets contain an introduced cDNA molecule that can be the molecular target of the perturbation. When a cell(s) is (are) identified the introduced cDNA molecules encode or are part of the a pathway the mediates the effect.

Accordingly, methods and products for rapidly identifying cellular targets of any molecule, such as a small molecule effector, that is biologically active, are provided. Thus, a genetic screening methodology for rapid identification of candidate targets of any cellular effector, such as a small molecule, is provided

Also provided are methods for identifying a nucleic acid molecules, such as cDNA molecules that, when expressed in a cell, cause an altered response of the cell, which alteration can be assessed by comparison with a control, such as a control cell. The response includes any detectable changed that can be induced or caused by an signal, including exposure of the cell to conditions, such as exposure to a biologically active molecule, that result in a response.

Such methods include the steps of: (a) providing a plurality of reporter cells that each cell contain a reporter construct that includes a nucleic acid molecule, such as cDNA, operably linked to a promoter such that the linked nucleic acid is expressed in the reporter cell; different linked nucleic acid molecules are expressed in each of the reporter cells; (b) exposing the reporter cells to a perturbation, such as contacting the reporter cells with a biologically active molecule; and (c) identifying a reporter cell or cells that has (have) an altered response (altered phenotype) to the perturbation, compared to a control, such as the same cell in the absence of the condition or in the absence of the reporter or in the presence of a condition with a known response. The introduced nucleic acid molecules can be a collection, such as a cDNA library or a tranascriptome, or RNA or antisense oligonucleotides in which each member of the collection is introduced into a each of an addressable collection of reporter cells, such as cells in an addressable array. The cells are screened to identify one or more nucleic aicd molecules that when added to the cell in some manner modulate (increase, decrease, or otherwise change) the response of a cell. The phenotype of the cells can be assessed.

In other embodiments, the nucleic acid can be added to the cell in the presence of or before or after the cells are exposed to a perturbation, such as a biologically active molecule, to which cells normally respond. Such nucleic acids, for example, can encode polypeptides that are cellular targets for the bioactive molecule, such as a receptor for which the bioactive molecule is an agonist, antagonist, or inverse agonist, for example. Alternatively, the cDNA can encode polypeptides that indirectly increase or decrease levels of the cellular target, such as target that is a polypeptide, lipid, nucleic acid, carbohydrate, factor or co-factor or other molecule or cellular target).

In other embodiments, the introduced nucleic acid molecule, such as cDNA, can encode a mutant, such as a truncated product, point mutation, deletional or insertional mutant, form of a gene that directly or indirectly produces a cellular target for the bioactive molecule.

As indicated above, one way to modulate the effect of a bioactive molecule is by overexpressing a cDNA in a reporter cell, thereby producing more of a target for the bioactive molecule, whether directly (the polypeptide encoded by the cDNA is itself a target for the bioactive molecule) or indirectly (for example, the polypeptide encoded by the cDNA is directly or indirectly responsible for production of the target for the bioactive molecule). Another way to modulate the effect of a bioactive molecule is to reduce amounts of its target. This can be accomplished, for example, by expression of a cDNA in an antisense orientation or by co-suppression or using siRNA or RNAi, for example. Another way to modulate the effect of a bioactive molecule is to express a mutant form of a cNDA, whether a truncated version of the cDNA, cDNA having various point mutations, etc.

The methods provided herein for a particular molecular target/small molecule pair, combines the ability to measurably modulate (increase, decrease, or otherwise affect) the biological effect of a small molecule by over-expression of its target in cells, with the utility of laboratory automation and arrayed cDNA expression library formats to identify targets efficiently.

The effect of a small molecule can be modulated by over-expression of its cellular target and measured using engineered cellular reporter gene assays. To exemplify this approach, as discussed in the Examples below, NF-κB dependent reporter cell lines were established in Jurkat T lymphocytes and HEK293 cells using a novel sin retroviral reporter termed S1N1. Salicylate, a known bioactive small molecule inhibitor of the kinase IKK-beta was shown to block TNF induction of NF-κB in both reporter cell types. Compared to controls, over-expression of cDNA encoding human IKK-beta diminished the inhibitory effects of salicylate on the NF-κB reporter in both cell types, either by transient over-expression in HEK293-derived reporter cells, or by stable retroviral over-expression in Jurkat reporter cells.

The ability to screen for cDNA that encodes cellular targets for effector action (or polypeptides that are responsible for directly or indirectly generating such targets) can identify additional targets for drug discovery, for example, by identifying members of biochemical pathways and identifying other factors that influence a given cellular process. In addition, the methods provided herein can determe the order of members of a biochemical pathway. By following an iterative process of identifying targets of small molecule effectors, then discovering small molecules that interact with such a target, and so on, biochemical pathways are mapped. In addition, the processes can be automated, significantly increasing the speed of the process and reducing its cost.

Exemplary of the uses for the arrays of reporter cells are their use to assess phenotypic changes resulting from the introduction of collections of nucleic acid molecules, including cDNA, antisense nucleic acids, dsRNAi, RNAi, siRNA, and other nucleic acid molecule whose expression or interaction with cellular nucleic acids alters gene expression (transcription and/or translation) or gene product activity. The collections of nucleic acis are contacted with the collections or reporter cells and any cells that exhibit phenotypic changes are identified (annotated).

In other embodiments the collections of nucleic acid molecules, including cDNA, antisense nucleic acids, dsRNAi, RNAi, siRNA, and other nucleic acid molecule whose expression or interaction with cellular nucleic acids alters gene expression (transcription and/or translation) or gene product activity are introducted simultaneously, before or after a the cells are exposed to a perturbation, such as condition or small effector molecule or other modulator of activity. Any cells that exhibit phenotypic changes and/or in which the phenotypic changes caused by either the perturbation condition or the introduced nucleic acid molecule are identified.

C. Preparation of Reporter Cells

Reporter cells are any cells that generate a detectable output representative of a particular cellular activity, function, pathway or inhibition thereof. As noted above, the activities that can be monitored include but are not limited to, gene expression, cell differentiation, cell proliferation, nuclear transport, protein trafficking, trafficking of other molecules into the cell or compartments thereof and other such processes.

Exemplary of the cellular output contemplated herein is gene expression in which a expression reporter, such as a detectable protein or an enzyme is operatively linked to a regulatory region that is in the pathway of interest. One such pathway and use of the methods herein to identify targets of small molecules is provided in the Examples.

1. Preparing Reporter Gene Constructs and Selection of Vectors

a. Isolation of Regulatory Regions

A regulatory regions, such as a promoter region, from a gene in a pathway of interest are identified, isolated, linked to reporter genes and introduced into cells, such as by insertion into a vector that can infect, transfect or transduce selected cells. The regulatory region is identified and isolated by standard molecular biology techniques, and cloned into a reporter constructs.

1) Identification of Inducibly Regulated Promoters

Regulatory elements that control transcription of a gene include the promoter region for the gene. Promoter regions and other transcriptional regulatory regions are usually 5‘or upstream of the gene’s coding sequence. The typical eukaryotic promoter includes a transcription initiation site, a binding site (TATA box), initiator, minimal or core promoter, proximal promoter region, and sometimes enhancer, silencer or locus control regions. Normally, sequences 1 to 10 kilobases (kB) upstream of the genes transcriptional start site contain all regulatory regions. Hence, upon identification of an inducible gene, selection of the region about 1 to 10 kB upstream thereof will contain regulatory regions of interest herein.

Identification of an inducible gene by methods herein or other such method permits identification of such regions. These regions can be identified by cloning and sequencing if necessary, and generally by searching public or proprietary databases for sequences identical to the gene of interest. Upon identification of the gene, the 5′ start site (methionine) of the gene and about 10 kB pair sequence upstream is identified. This 10 kB sequence generally contains a promoter region controlling expression of the gene of interest. This analysis is enhanced by searching for consensus promoter regions, or transcription factor binding motif sequences or enhancer elements.

Based upon the identity of the responder gene, the regulatory region is then identified. Identification of candidate regulatory region, such as a promoter-containing region, for any gene can be done by any method known to those of skill in the art, including manually and/or by database searching. For example, following identification of a gene whose expression increases or decreases in the presence of a test substance or stimulus, a regulatory region of the gene can be identified by probing genomic sequences, such as a genomic library) with the gene or fragment thereof for hybridizing sequences that also include 5′ or 3′ untranslated sequences of the gene.

Alternatively, RNA extension (to identify the transcriptional start site) followed by genomic DNA “primer walking” to identify sequences upstream of the transcription start site can be used. These methods are standard and well known in the art (see, e.g., Sambrook et al. (1989) Molecular Cloning: A Laboratory Manual (2nd Ed.), Vol. 1-3, Cold Spring Harbor Laboratory, Cold Spring Harbor, N.Y.).

Candidate gene regulatory regions can be identified by comparison of the gene to a sequence database available in the art now or in the future. For example, a public or proprietary sequence database that includes genomic sequence information can be used to identify sequences located 5′ or 3′ of the translation initiation site of the selected gene, as well as intron(s). Because sequences located 5′ and extending upstream of the translation initiation site frequently contain gene regulatory sequences, nucleotide sequences positioned 5′ of the translation initiation site are good candidates for regulatory sequences and can be selected for cloning into a reporter construct. For example, a sequence that includes the 5′ translation start site (methionine) of the gene and 10 Kb or more upstream of the site contains intronic and exonic portions of the gene, but likely also the promoter region controlling expression of the gene. The embodiment of database searching for selecting candidate gene regulatory regions is exemplified in Example 3.

Sequence databases of any organism can be searched in order to identify candidate regulatory regions. Partial and complete sequence databases of many organisms, including mammals, are available in the art. Databases are available and can be found using any suitable internet search engine to identify sites posting such databases (see, e.g., www.ncbi.nlm.nih.gov/genome/seq/page.cgi?F=HsBlast.html&&ORG=Hs for a human database. Other human databases are available for a fee, such as the database owned by Celera, Inc. Similarly, mouse partial genomic sequences are available (see, e.g., http://www.ncbi.nim.nih.gov/genome/seq/MmHome.html). The complete yeast Saccharomyces cerevisiae genomic sequence is available (see, e.g., http:/lwww.ncbi.nlm.nih.gov/cgi-bin/Entrez/map00?taxid=4932). In addition, the complete Drosophila melanogaster and C. elegans genomic databases are known in the art (see, e.g., http://www.ncbi.nim.nih.gov/PMGifs/Genomes/7227.html and http://www.ncbi.nlm.nih.gov/cgi-bin/Entrez/map00?taxid=6239). Plant databases include, for example, the complete sequence of Arabidopis thaliana (see, e.g., http://www.ncbi.nim.nih.gov/cgi-bin/Entrez/map_search?chr=arabid.inf). It is understood that URLs for the databases can change and particular information on the internet can come and go, but equivalent information can be found by searching the internet.

Sequence database analysis can be augmented, if desired or needed, by searching for consensus promoter regions, transcription factor binding sequences or enhancer elements. For example, inspecting a gene for a candidate regulatory region can reveal a known regulatory region or a sequence having significant similarity with a known regulatory region. Thus, including a search for one or more sequences homologous or having significant similarity to a known promoter, transcription factor binding site or enhancer can reveal the presence and location of such sequences in the genomic sequence which can then be cloned into the reporter expression construct. Thus, methods herein can be modified to include the strep of identifying regulatory regions by comparison to other regulatory region sequences, such as known regulatory region sequences, including, but not limited to sequences including promoters, transcription factor binding sites, enhancers, scaffold attachment regions and other such transcription and/or translational regulatory regions.

Candidate regulatory regions can be of any length so long as expression in response to the test substance or stimulus is at least in part reflective of expression in the original screen. In other words, expression of a reporter driven by the selected regulatory region need not precisely mirror expression of the endogenous gene in response to the substance or stimulus. In any event, significant variation between endogenous gene expression and reporter gene expression can be minimized by including larger portions of the candidate regulatory region sequence in the reporter construct. Thus, when first choosing a sequence of a candidate regulatory region for cloning into a reporter, larger sequences can be selected. Candidate regulatory regions can therefore include large sequences such as 10,000-15,000 nucleotides or more, 5000-10,000 nucleotides, 1000-5000 nucleotides, and 50-5000 nucleotides.

Inspecting a gene for consensus promoters, transcription factor binding sites, enhancers and other sequences can reveal the presence of one or more such sequences or a sequence that exhibits significant sequence homology to a consensus sequence. When such a consensus sequence is present, a smaller region of the candidate regulatory region that includes the consensus sequence can be chosen for subsequent cloning into a reporter construct. Of course, should there be multiple consensus sequences in the candidate cis-acting regulatory region of a gene, a sequence can be chosen that includes two or more of the multiple consensus sequences. Candidate regulatory regions can therefore include smaller sequences, for example, 50-5000 nucleotides, such as about 5-10, 10-25, 25-50, 50-75, 75-100, 100-250, 250-500, 1000-2500, or 2500-5000 nucleotides.

The untranslated region/candidate regulatory region can subsequently be cloned into a reporter expression construct and introduced into cells. Expression of the reporter in the presence and absence of the test substance or stimulus confirms that the cloned region contains all or at least a part of the regulatory region that mediates the response to the test substance or stimulus.

Repeating the steps of identifying or selecting responder genes and cloning a regulatory region therefrom operatively linked to a reporter produces collections of gene regulatory region-reporter constructs (i.e., a library). The accumulation of collections of gene regulatory regions, and reporter constructs containing gene regulatory regions of the entire complement of an organism (e.g., human gene promoters) would be a highly useful resource.

Methods of producing a plurality of gene regulatory regions, such as a library, compositions containing the gene regulatory regions produced by the methods, as well as methods of producing a plurality of gene regulatory region-reporter constructs and compositions containing a plurality of gene regulatory region-reporter constructs produced by the methods. In one embodiment, the plurality contains gene regulatory region-reporter constructs in which expression of the reporter is increased at least three-fold in the presence of the test substance or stimulus in comparison to the absence of the test substance or stimulus. In another embodiment, the plurality contains gene regulatory region-reporter constructs in which expression of the reporter is decreased at least six-fold in the presence of the test substance or stimulus in comparison to the absence of the test substance or stimulus.

2) Extraction and Cloning of Regulatory Regions, Such as Promoters

The following methodology was used to extract promoter regions from a sequence database and can be generally applied to any DNA sequence database: Unigene, downloaded from NCBI, was parsed for entries where the coding region is explicitly defined (currently 18289 such entries exist). Three hundred bases from the 5′ end of each coding region are assembled into a FASTA file. This file is then aligned to genomic sequence using the BLAST algorithm. The target genomic database can be NR or HTGS from NCBI, or the Celera genome assembly. The BLAST alignments are parsed to determine the location of the gene in a larger genomic contig, and up to 10 kb of sequence is taken upstream of the translational start site. Several 1000 promoter sequences have been assembled in silico using this technique.

Genomic DNA is prepared from Human 293 cells using DNAzol. Oligonucleotide primers are synthesized from 20, two kB promoter sequences at a time. Polymerase chain reaction (PCR) is used to amplify promoter sequences from chromosomal DNA templates and cloned into standard reporter gene constructs in which the cloned promoter drivers expression of the Firefly Luciferase (luc) gene or some other reporter gene. The DNA encoding each promoter reporter construct is individually amplified in bacterial cells and purified in micro-titer plates using a Rev-Prep (Molecular Machines) or Qiagen 9600 (Qiagen). Ninety-six well plates of reporter constructs are re-racked into 384-well plates for subsequent use such that each 384-well plate has 4 wells of each reporter construct.

Regulatory regions can be identified by their presence 5′ from a translation initiation site of the gene, within or a part of the gene coding sequence (e.g., within exons), within or be a part of non-coding intragenic sequences (e.g., introns) or located 3′ of the translation stop site. Candidate regulatory regions can therefore be located throughout a genomic sequence, including sequences within 25 bases, 50 bases, 100 bases, 250 bases, 500 bases, 1 Kb, 2 Kb, 3 Kb, 4 Kb, 5 Kb, 7 Kb, 10 Kb, 15 Kb or more from the translation initiation site and translation termination site of a gene. Hence the location of the gene regulatory region relative to the gene coding sequence is not fixed.

For example, a sequence located 5′of the translation start site can be cloned into the reporter construct. Longer sequence segments of the candidate regulatory region (e.g., 30 Kb, 20 Kb, 10 Kb, or 5 Kb) can first be examined for conferring increased or decreased reporter expression. Smaller segments can then be examined, if desired, in order to identify smaller segments that confer regulation. A segment of the genomic sequence is cloned (using polymerase chain reaction, conventional restriction enzyme cloning or chemical synthesis) into a reporter construct so that reporter expression is controlled by the segment.

Thus, a regulatory region is located 5′ of the gene coding region and extends upstream of the translation initiation site. The regulatory region can include a promoter or enhancer and can be located in or as part of one or more exons, one or more introns or 3′ of the gene coding region and extending downstream of the translation termination site. In particular aspects, the sequence region extends from about 25, 50, 75, 100, 250, 500, 1000, 2500, 5000, 7500 or 10,000 or more nucleotides upstream of the translation initiation site of the selected gene. In particular additional aspects, the sequence region extends from about 25, 50, 75, 100, 250, 500, 1000, 2500, 5000, 7500 or 10,000 or more nucleotides downstream of the translation termination site of the selected gene.

b. Reporters and Reporter Gene Constructs

Following selection of a regulatory region, based on examination or cloning of genomic sequence with or without inspecting for the presence of consensus regulatory regions or sequences with similarity to such regions (e.g., promoter sequences, transcription factors binding sequences, enhancer sequences, silencers and others), the sequence can be cloned into a reporter expression construct. Operatively linking a sequence including a 5′ untranslated region upstream of the translation initiation site or any other candidate regulatory region of the selected gene to a reporter gene and determining reporter expression in the presence of the test substance or stimulus confirms that the sequence mediates the response to the test substance or stimulus.

Reporter gene constructs include a reporter gene such as the nucleic acid encoding firefly luciferase, Renilla luciferase and the aqueorin photoprotein and mutants thereof, beta-galactosidase, a fluorescent protein, secreted alkaline phosphatase, chloramphenicol acetyltransferase or other element under the control of a response-element such as a promoter sequence from the robust responder gene. Reporter moieties also include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyltransferase (CAT) and other such well-known reporters.

c. Vectors and Generation of Viral Particles and Reporter Cells Containing the Reporter Gene Constructs

The vector constructs are used to generate recombinant viral particles and to transfect, either transiently or stably, suitable eukaryotic, typically mammalian, host cells. To generate viruses using the construct described above, retroviral producer cells, either stably derived or transients created by short-term expression of retroviral packaging components, such as structural and functional proteins (i.e., gag-pol and env expression constructs) are plated out for subsequent generation of viral particles encoding the reporter construct. These cells are transfected with the retroviral reporter construct by any suitable method, including direct uptake, calcium phosphate precipitation, lipid-mediated delivery, such as LipofectAMINE (Life Technologies, Burlington, Ont., see U.S. Pat. No. 5,334,761), or any DNA delivery vehicle. Once the DNA enters cells, the cells provide the proteins for production of RNA and packaging of the RNA into the retroviral particles. The virus is released into the supernatant and harvested.

The viral supernatant is applied to a target population of cells, typically the cells from which the inducible promoter was originally identified, and incubated. The cells are treated to permit the viruses to enter the cells (transduce) convert the RNA reporter construct to DNA (via reverse transcription) and integrate into the chromatin of the target cells. Once integrated, since the reporter vector is “SIN”, the promoter regions in the U3 are no longer present and the only promoter remaining is that inserted upstream of the reporter gene.

Cells infected with the virus can be selected with agents that eliminate untransduced cells, identify transduced cells, or some method that exploits the “marker” gene to detect transduced cells. In this way, a population of cells expressing the reporter construct is isolated. The marker also can be used to determine the efficiency of viral transduction. Once selected, the cells are treated with the substance or stimulus originally used to identify the inserted regulatory region(S). Studies are performed to recapitulate the magnitude of change experienced by genes under control of the promoter to confirm that the appropriate regulatory region is present in the reporter. If a response that originally observed in the gene expression array screen is not seen at least in part, clones, or individually transduced cells can be isolated and tested to isolate stronger responders. The thus identified and isolated cell(s) constitute the reporter cells. For the methods herein, a particular regulatory region is selected and cells containing the regulatory region linked to the reporter are exposed to modulators, including small molecules, genes, and various signals, such as molecular entities, that perturb cell function, particularly those that modulate or effect or affect regulation of the regulatory region, including the promoter, of the selected output and nucleic acid encoding potential targets for the modulator.

Vectors for introducing the reporter constructs include, but are not limited to, any that are appropriate for conferring expression in any prokaryotic or eukaryotic organism for which a cell that expresses a reporter driven by a gene regulatory region of an organism, cell type, tissue, organ or other selected cell source. Exemplary organisms include animals, such as mammals including humans, bacteria, yeast, parasites, insects and plants. Vectors for use in these and other organisms are well known in the art. For example, for mammals, virus vectors include adeno- and adeno-associated virus (U.S. Pat. Nos. 5,700,470, 5,731,172 and 5,604,090), polyoma virus, retrovirus (see, e.g., U.S. Pat. Nos. 5,624,820, 5,693,508 and 5,674,703; and International PCT application No. WO 92/05266 and WO92/14829; lentiviral vectors are described, e.g., in U.S. Pat. No. 6,013,516), papilloma virus (see, e.g., U.S. Pat. No. 5,719,054), herpes simplex virus vectors (see, e.g., U.S. Pat. No. 5,501,979), CMV-based vectors (see, e.g., U.S. Pat. No. 5,561,063), semiliki forest virus, rhabdovirus, parvovirus, picornavirus, reovirus, lentivirus, rotavirus, simian virus 40 and others.

For insects, baculovirus vectors can be used; for yeast, yeast artificial chromosomes or self-replicating 2 μm (e.g., YEp) or centromeric (e.g., YCp) based vectors can be used; for bacteria, pBR322 based plasmids can be used; for plants, CaMV based vectors can be used. See, e.g., Ausubel et al. (1988) In: Current Protocols in Molecular Biology, Vol. 2, Ch. 13, ed., Greene Publish. Assoc. & Wiley Interscience; Grant et al. (1987) In: Methods in Enzymology, 153:516-544, eds. Wu & Grossman, 31987, Acad. Press, N.Y.; Glover, DNA Cloning, Vol. II, Ch. 3, IRL Press, Wash., D.C., 1986; Bitter (1987) In: Methods in Enzymology 152:673-684, eds. Berger & Kimmel, Acad. Press, N.Y.; and, Strathern et al. (1982) The Molecular Biology of the Yeast Saccharomyces, Cold Spring Harbor Press, Vols. I and II; Rothstein (1986) in: DNA Cloning, A Practical Approach, Vol.11, Ch. 3, ed. D. M. Glover, IRL Press, Wash., D.C.; Goeddel (1990), Gene Expression Technology: Methods in Enzymology 185, Academic Press, San Diego, Calif.; Brisson et al. (1984) Nature 310:511; Odell et al. (1985) Nature 313:810). Vectors can include a selection marker. As is known in the art, “selection marker” means a gene that allows selection of cells containing the gene. “Positive selection” means that only cells that contain the selection marker will survive upon exposure to the positive selection agent. For example, drug resistance is a common positive selection marker; cells containing a drug resistance gene will survive in culture medium containing the selection drug; whereas those which do not contain the resistance gene will die. Suitable drug resistance genes are neo, which confers resistance to G418, hygr, which confers resistance to hygromycin and puro, which confers resistance to puromycin. Other positive selection marker genes include reporter genes that allow identification by screening of cells.

These genes include genes for fluorescent proteins (GFP), the lacZ gene (β-galactosidase), the alkaline phosphatase gene, and chlorampehnicol acetyl transferase. Vectors provided herein can contain negative selection markers.

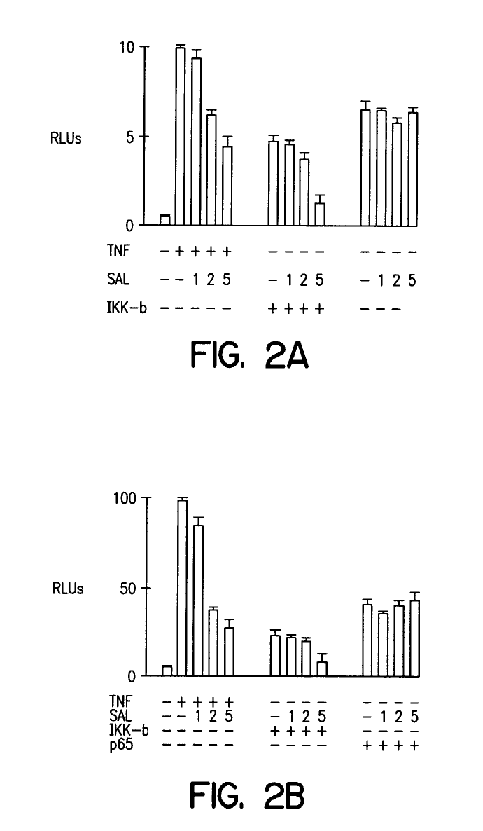

Vectors of particular interest herein are retroviral vectors. Retroviral vectors can be introduced into a large variety of host cells with high transduction efficiencies. FIG. 2 sets forth retroviral transduction efficiencies for exemplary cell types and cellular processes that can be studied using each cell type. A large number of retroviruses have been developed and are well known. Such vectors include, but are not limited to, moloney murine leukemia virus (MoMLV) and derivatives thereof, such as MFG vectors (see, e.g., U.S. Pat. No. 6,316,255 B1, ATCC acession No. 68754); myeloproliferative sarcoma virus (MPSV), murine embryonic stem cell virus (MESV), murine stem cell virus (MSCV), lentivirus vectors (HIV and FIV vectors), spleen focus forming virus (SFFV); MSCV retroviral vectors, and many others. Retroviral vectors are designed to deliver nucleic acid to a cell and integrate into a chromosome, but are designed so that they lack elements necessary for productive infection.

One exemplary retroviral vector contemplated for use herein is a self-inactivating (SIN) retrovirus. As noted above, self-inactivating retroviruses have the 3′LTR and U3 regions removed so that upon recombination the LTR is gone A functional U3 region in the 5′ LTR permits expression of a recombinant viral genome in appropriate packaging lines. Upon expression of its genomic RNA and reverse transcription into cDNA, the U3 region of the 5′ LTR of the original provirus is deleted and replaced with defective U3 region of the 3′ LTR. As a result, when a SIN vector integrates, the non-functional 3′ LTR replaces the functional 5′ LTR U3 region, rendering the virus incapable of expressing the full-length genomic transcript.

A viral vector can additionally include a scaffold attachment region (SAR) for circumventing cis-effects of integration on promoter activity; a unidirectional transcription blocker (utb) to avoid competitive transcription; or a selectable or detectable marker. The efficiency afforded by use of these elements (SIN, SAR, utb, selection/detection cassette) for developing reporter gene assays allows rapid analysis of gene regulatory regions.

Thus, also provided are viral expression vectors. In one embodiment, a viral vector with a unidirectional transcriptional blocker and a selectable or detectable marker, or a reporter is provided. In another embodiment, a viral vector can include a scaffold attachment region and a selectable or detectable marker, or a reporter. In yet another embodiment, a viral vector can contain a unidirectional transcriptional blocker, a scaffold attachment region and a selectable or detectable marker, or a reporter. In still another embodiment, a viral vector can include a unidirectional transcriptional blocker, a scaffold attachment region and a selectable or detectable marker, and a reporter. In one aspect, the viral vector is a retroviral vector. In one particular aspect, the retroviral vector has a mutated or deleted LTR so that the vector is self-inactivating.

An exemplary retroviral vector contains the following characteristics: a promoter/enhancer region (LTR, or U3RU5) at the 5′ end; a deleted portion of the 3′ LTR so that the promoter/enhancer function of the LTR is mutated or deleted (SIN, or self-inactivating vector); a psi (ψ) sequence for packaging the vector into a retroviral particle or virion; a region for insertion of a candidate regulatory region (denoted “PROMOTER”), with the upstream promoter sequence being oriented at the 3′ end of this vector, and the downstream portion being oriented at the 5′ end of the vector; a reporter such as a luciferase, including firefly luciferases and Renilla luciferases, beta-galactosidase, fluorescent proteins (FPs), such as (green, red and blue FPs), secreted alkaline phosphatase, chloramphenicol acetyltransferase, lacZ; a scaffold attachment region (SAR) or a sequence that reduces or prevents nearby chromatin or adjacent sequences from influencing this promoter's control of the reporter gene; a constitutive promoter “pro” (such as phosphoglucokinase, actin, or SV40) driving a selectable marker (such as an antibiotic resistance gene, fluorescent, luminescent, colorimetric gene) or gene conferring a selective advantage to cells expressing it; a unidirectional transcriptional blocker (utb) sequence between the marker gene and reporter gene; a “U3” region at the 5′ end not normally found in retroviruses to increase expression, viral titers and thus efficient delivery of the completed reporter gene to cells.

Retroviral expression vector reporter constructs are provided herein that includes one or more of the following characteristics or elements:

1) a promoter/enhancer region (LTR or U3RU5) at the 5′ end;

2) a deleted portion of the 3′ LTR, wherein the U3 region, which contains the promoter/enhancer function of the LTR, is mutated or deleted (to produce a SIN, or self-inactivating vector);

3) a psi (ψ) sequence for packaging the RNA genome derived from the vector in cells into a retroviral particle or virion;

4) an inducible promoter of interest (PROMOTER) with, for example, a polylinker inserted in this region for cloning, with the upstream promoter sequence oriented at the 3′ end of this vector, and the downstream portion oriented at the 5′ end of the vector so that in the DNA vector the relation of the promoter to the “reporter” gene is identical to that of the promoter to the actual gene it regulates in the human genome;

5) a selectable marker or reporter, such as, but are not limited to, firefly luciferase, Renilla luciferase, beta-galactosidase, green, blue and/or red fluorescent protein, secreted alkaline phosphatase and combinations thereof, as described above;

6) a scaffold attachment region (SAR) or a sequence or member of a family of sequences (such sequences can be found in the interferon-beta gene (IFN-beta) and are also called insulators; see U.S. Pat. No. 6,194,212) that constrict nearby chromatin, or adjacent sequences from influencing the promoter's control of the reporter gene;

7) a constitutive promoter “pro” (such as, but are not limited to, phosphoglucokinase, actin, and SV40 promoter) controlling expression of a selectable marker or reporter (such as an antibiotic resistance gene, fluorescent, luminescent, calorimetric gene) or gene conferring a selective advantage to cells expressing it, thereby permitting differentiation or isolation of only those cells expressing it;

8) a unidirectional transcriptional blocker (utb) sequence between the marker gene and reporter gene such that marker genes transcribed from the “pro” terminate transcription at some efficiency after the marker to avoid interfering with expression from the “PROMOTER” and the reporter gene transcript RNA, such as via an antisense competition mechanism; and

9) a “U3” region at the 5′ end not normally found in retroviruses, such as a CMV, RSV or other strong constitutive promoter/enhancer sequences to provide for high levels of expression, viral titers and thus efficient delivery of the completed reporter gene to cells.

The structure of the vector can be represented as follows: U3* R U5 ψ pro marker utb reporter PROMOTER SAR ΔU3 R U5, where the order of certain elements, such as the SAR whose effect is position independent, can be changed.

Any retroviral and other sources of these components can be employed. Retroviruses that can serve as sources of these retroviral sequences include, for example moloney murine leukemia virus (MoMLV), myeloproliferative sarcoma virus (MPSV), murine embryonic stem cell virus (MESV), murine stem cell virus (MSCV) and spleen focus forming virus (SFFV). The regulatory region (e.g., promoter) derived from gene chip or by other methods, or gene regulatory sequences are cloned into the PROMOTER region of the vector for generation of responder cells. The vectors are introduced into cells to produce a collection of reporter cells.

The plasmid pNFκB-Luc (available from Clontech, see, SEQ ID No. 3) contains four tandem copies of the NFκB consensus sequence fused to a TATA-like promoter (PTAL) region from the Herpes simplex virus thymidine kinase (HSV-TK) promoter. NF-κB binds to the κB4 element on the vector and initiates transcription of luciferase. After endogenous NFKB proteins bind to the kappa (κ) enhancer element (κB4), transcription of the pNFκB-luc is induced and the reporter gene, luciferase, is activated. The luciferase coding sequence is followed by the SV40 late polyadenylation signal to ensure proper, efficient processing of the luc transcript in eukaryotic cells. Located upstream of NFκB is a synthetic transcription blocker (TB), which is composed of adjacent polyadenylation and transcription pause sites for reducing background transcription (Eggermont et al., (1993) EMBO J. 12:2539-2548). The vector backbone also contains an f1 origin for single-stranded DNA production, a pUC origin of replication, and an ampicillin resistance gene for propagation and selection in E. coli. The plasmid pNFκB-Luc was designed to measure the binding of transcription factors to the enhancer, which provides a direct measurement of activation of this pathway. For example, the addition of TNFα, II-1, or other lymphokine receptors to a cell-culture medium induces the binding of transcription factors to the κ enhancer, which initiates transcription of the luciferase reporter gene. The reporter portion (regulatory region and luciferase encoding nucleic acid) of this plasmid has been introduced into retroviral vectors herein and introduced into cells as a means of monitoring this pathway and for exemplification of the methods herein.

For example, addition of inhibitors of this pathway (in the presence of agonist) will prevent expression of the reporter gene, and addition of nucleic acids that are or encode the target of the inhibitors will restore expression of the reporter gene and thereby permit identification of targets.

2. Recombinase Systems

Recombinase systems provide an alternative way to generate arrays of reporter cells. Recombinases are used to introduce the reporter gene constructs into chromosomes modified by inclusion of the appropriate sequence(s) for recombination in the cells. Site specific recombinase systems typically contain three elements: two pairs of DNA sequences (the site-specific recombination sequences) and a specific enzyme (the site-specific recombinase). The site-specific recombinase catalyzes a recombination reaction between two site-specific recombination sequences.

A number of different site specific recombinase systems are available and/or known to those of skill in the art, including, but not limited to: the Cre/lox recombination system using CRE recombinase (see, e.g., SEQ ID Nos. 5 and 6) from the E. coli phage P1 (see, e.g., Sauer (1993) Methods in Enzymology 225:890-900; Sauer et al. (1990) The New Biologist 2:441-449), Sauer (1994) Current Opinion in Biotechnology 5:521-527;; Odell et al. (1990) Mol gen Genet. 223:369-378; Lasko et al. (1992) Proc. Natl. Acad. Sci. U.S.A. 89:6232-6236; U.S. Pat. No. 5,658,772), the FLP/FRT system of yeast using the FLP recombinase (see, SEQ ID Nos. 7 and 8) from the 2μ episome of Saccharomyces cerevisiae (Cox (1983) Proc. Natl. Acad. Sci. U.S.A. 80:4223; Falco et al. (1982) Cell 29:573-584; (Golic et al. (1989) Cell 59:499-509; U.S. Pat. No. 5,744,336), the resolvases, including Gin recombinase of phage Mu (Maeser et al. (1991) Mol Gen Genet. 230:170-176; Klippel, A. et al (1993) EMBO J. 12:1047-1057; see, e.g., SEQ ID Nos. 9-12) Cin, Hin, αδ Tn3; the Pin recombinase of E. coli (see, e.g., SEQ ID Nos. 13 and 14) Enomoto et al. (1983) J Bacteriol. 6:663-668), and the R/RS system of the pSR1 plasmid of Zygosaccharomyces rouxii (Araki et al. (1992) J. Mol. Biol. 225:25-37; Matsuzaki et al. (1990) J. Bacteriol. 172: 610-618) and site specific recombinases from Kluyveromyces drosophilarium (Chen et al. (1986) Nucleic Acids Res. 314:4471-4481) and Kluyveromyces waltii (Chen et al. (1992) J. Gen. Microbiol. 138:337-345). Other systems are known to those of skill in the art (Stark et al. Trends Genet. 8:432-439; Utatsu et al. (1987) J. Bacteriol. 169:5537-5545; see, also, U.S. Pat. No. 6,171,861).

Members of the highly related family of site-specific recombinases, the resolvase family, such as γδ, Tn3 resolvase, Hin, Gin, and Cin) are also available. Members of this family of recombinases are typically constrained to intramolecular reactions (e.g., inversions and excisions) and can require host-encoded factors. Mutants have been isolated that relieve some of the requirements for host factors (Maeser et al. (1991) Mol. Gen. Genet. 230:170-176), as well as some of the constraints of intramolecular recombination (see, U.S. Pat. No. 6,171,861).

The bacteriophage P1 Cre/lox and the yeast FLP/FRT systems are particularly useful systems for site specific integration or excision of heterologous nucleic acid into chromosome. In these systems a recombinase (Cre or FLP) interacts specifically with its respective site-specific recombination sequence (lox or FRT, respectively) to invertor excise the intervening sequences. The sequence for each of these two systems is relatively short (34 bp for lox and 47 bp for FRT).

The FLP/FRT recombinase system has been demonstrated to function efficiently in plant cells (U.S. Pat. No. 5,744,386), and, thus, can be used for plants as well as animal cells. In general, short incomplete FRT sites leads to higher accumulation of excision products than the complete full-length FRT sites. The system catalyzes intra- and intermolecular reactions, and, thus, can be used for DNA excision and integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g., U.S. Pat. No. 5,658,772). This system is used to excise a gene located between two lox sites. Cre is expressed from a vector. Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

Any site-specific recombinase system known to those of skill in the art is contemplated for use herein. It is contemplated that one or a plurality of sites that direct the recombination by the recombinase are introduced into chromosomes, and then heterologous genes linked to the cognate site are introduced into chromosomes. The E. coli phage lambda integrase system can be used to introduce heterologous nucleic acid into chromosomes (Lorbach et al. (2000) J. Mol. Biol 296:1175-1181). For purposes herein, one or more of the pairs of sites required for recombination are introduced into a chromosome. The enzyme for catalyzing site directed recombination can be introduced with the DNA of interest, or separately.

D. Methods for the Delivery of Nucleic Acids into Cells

A variety of methods for delivering nucleic acids into cells are known. Such methods, include, but are not limited to electroporation, sonoporation, direct uptake, such as by calcium phosphate precipitation, lipofection, by microcell fusion, lipid-mediated carrier systems, other suitable methods, and combinations of any such methods. The method selected for delivering particular nucleic acid molecules, such as DNA, to targeted cells can depend on the particular nucleic acid molecule being transferred and the particular recipient cell and can be determined empirically using methods known to those of skill in the art.

Exemplary methods for introducing a plurality of nucleic acids into collections of cells are known (see, e.g., Ziauddin et al. (2001) Nature 411:107-110, and published International PCT application No. W0 01/20015; see also published U.S. application Serial No. US2002000664A1.

Delivery Agents and Treatments

Delivery agents include compositions, conditions and physical treatments that permit introduction of nucleic acids into cells. Such agents and treatments include, but are not limited to, cationic compounds, peptides, proteins, energy, for example ultrasound energy and electric fields, and cavitation compounds. For example, compounds and chemical compositions, including, but not limited to, calcium phosphate, DMSO, glycerol, chloroquine, sodium butyrate, polybrene and DEAE-dextran, peptides, proteins, temperature, light, pH, radiation and pressure can be used. Other agents, such as as cationic compounds also are contemplated.

Cationic Compounds

Cationic compounds for use in the methods provided herein are available commercially or can be synthesized by those of skill in the art. Any cationic compound can used for delivery of nucleic acid molecules, such as DNA, into a particular cell type using the provided methods. One of skill in the art by using suitable screening procedures can readily determine which of the cationic compounds are best suited for delivery of specific nucleic acid molecules, such as DNA, into a specific target cell type.

(a) Cationic Lipids

Cationic lipid reagents can be classified into two general categories based on the number of positive charges in the lipid headgroup; either a single positive charge or multiple positive charges, usually up to 5. Cationic lipids are often mixed with neutral lipids prior to use as delivery agents. Neutral lipids include, but are not limited to, lecithins; phosphotidylethanolamine; phosphatidylethanolamines, such as DOPE (dioleoylphosphatidylethanolamine), DPPE (dipalmitoylphosphatidylethanolamine), dipalmiteoylphosphatidylethanolamine, POPE (palmitoyloleoylphosphatidylethanolamine) and distearoylphosphatidylethanolamine; phosphotidylcholine; phosphatidylcholines, such as DOPC (dioleoylphosphidylcholine), DPPC (dipalmitoylphosphatidylcholine) POPC (palmitoyloleoylphosphatidylcholine) and distearoylphosphatidylcholine; fatty acid esters; glycerol esters; sphingolipids; cardiolipin; cerebrosides; and ceramides; and mixtures thereof. Neutral lipids also include cholesterol and other 3βOH-sterols.

Other lipids contemplated herein, include: phosphatidylglycerol; phosphatidylglycerols, such as DOPG (dioleoylphosphatidylglycerol), DPPG (dipalmitoylphosphatidylglycerol), and distearoylphosphatidylglycerol; phosphatidylserine; phosphatidylserines, such as dioleoyl- or dipalmitoylphosphatidylserine and diphosphatidylglycerols.

Examples of cationic lipid compounds include, but are not limited to: Lipofectin (Life Technologies, Inc., Burlington, Ont.)(1:1 (w/w) formulation of the cationic lipid N-N,N,N-trimethylammonium chloride (DOTMA) and dioleoylphosphatidylethanolamine (DOPE)); LipofectAMINE (Life Technologies, Burlington, Ont., see U.S. Pat. No. 5,334,761) (3:1 (w/w) formulation of polycationic lipid 2,3-dioleyloxy-N-N,N-dimethyl-1-propanaminiumtrifluoroacetate (DOSPA) and dioleoyl phosphatidylethanolamine (DOPE)), LipofectAMINE PLUS (Life Technologies, Burlington, Ont. see U.S. Pat. Nos. 5,334,761 and 5,736,392; see, also U.S. Pat. No. 6,051,429) (LipofectAmine and Plus reagent), LipofectAMINE 2000 (Life Technologies, Burlington, Ont.; see also International PCT application No. WO 00/27795) (Cationic lipid), Effectene (Qiagen, Inc., Mississauga, Ontario) (Non liposomal lipid formulation), Metafectene (Biontex, Munich, Germany) (Polycationic lipid), Eu-fectins (Promega Biosciences, Inc., San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃₀.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER (Gene Therapy Systems Inc., San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

(b) Non-Lipid Cationic Compounds

Non-lipid cationic reagents include, but are not limited to SUPERFECT™ (Qiagen, Inc., Mississauga, ON) (Activated dendrimer (cationic polymer:charged amino groups) and CLONfectin™ (Cationic amphiphile N-t-butyl-N′-tetradecyl-3-tetradecyl-aminopropionamidine) (Clontech, Palo Alto, Calif.). Pyridinium amphiphiles are double-chained pyridinium compounds, which are essentially nontoxic toward cells and exhibit little cellular preference for the ability to transfect cells. Examples of a pyridinium amphiphiles are the pyridinium chloride surfactants such as SAINT-2 (1-methyl-4-(1-octadec-9-enyl-nonadec-10-enylenyl) pyridinium chloride) (see, e.g., van der Woude et al. (1997) Proc. Natl. Acad. Sci. U.S.A. 94:1160). The pyridinium chloride surfactants are typically mixed with neutral helper lipid compounds, such as dioleoylphosphatidylethanolamine (DOPE), in a 1:1 molar ratio. Other Saint derivatives of different chain lengths, state of saturation and head groups can be made by those of skill in the art and are within the scope of the present methods.

Energy

Delivery agents also include treatment or exposure of the cell and/or nucleic acid molecules, but generally the cells, to sources of energy, such as sound and electrical energy.

Ultrasound

For in vitro and in vivo transfection, the ultrasound source should be capable of providing frequency and energy outputs suitable for promoting transfection. Preferably, the output device can generate ultrasound energy in the frequency range of 20 kHz to about 1 MHz. The power of the ultrasound energy is preferably in the range from about 0.05 w/cm² to 2 w/cm², more preferably from about 0.1 w/cm² to about 1 w/cm². The ultrasound can be administered in one continuous pulse or can be administered as two or more intermittent pulses, which can be the same or can vary in time and intensity.

Ultrasound energy can be applied to the body locally or ultrasound-based extracorporeal shock wave lithotripsy can be used for “in-depth” application. The ultrasound energy can be applied to the body of a subject using various ultrasound devices. In general, ultrasound can be administered by direct contact using standard or specially made ultrasound imaging probes or ultrasound needles with or without the use of other medical devices, such as scopes, catheters and surgical tools, or through ultrasound baths with the tissue or organ partially or completely surrounded by a fluid medium. The source of ultrasound can be external to the subject's body, such as an ultrasound probe applied to the subject's skin which projects the ultrasound into the subject's body, or internal, such as a catheter having an ultrasound transducer which is placed inside the subject's body. Suitable ultrasound systems are known (see, e.g., International PCT application No. WO 99/21584 and U.S. Pat. No. 5,676,151).

Electroporation

Electroporation temporarily opens up pores in a cell's outer membrane by use of pulsed rotating electric fields. Methods and apparatus used for electroporation in vitro and in vivo are well known (see, e.g., U.S. Pat. Nos. 6,027,488, 5,993,434, 5,944,710, 5,507,724, 5,501,662, 5,389,069, 5,318,515). Standard protocols can be employed.

E. Preparation of Addressable Arrays of Cells Containing Heterologous Nucleic Acids

1. Nucleic Acid Transfer and Construction of cDNA Matrix

Nucleic acid solutions, such as miniprep DNA, are typically isolated and stored in a 96-well format. A portion of of this solution is transferred to a 384 (“master”) plate using conventional methods (i.e. Tecan, Hydra, etc.). Sub-microliter quantities (about 10, 20, 50 up to 1000 nanoliters) of the solutions are transferred in parallel from the master plate to tissue culture treated 384, 1536, or greater, well (“destination”) plates utilizing a “dry touch-off” (transfer of liquid onto a dry surface) procedure, which spots samples directly to the bottom of each well with minimal contamination between and among samples.

Delivery can be effected by any of the known methods and devices for delivering small volumes of samples using known delivery agents and treatments such as those described herein. For delivery to microtiter plates, such as 1536 well plates, the MiniTrak, manufactured by Packard can be used. Other such devices are known and commercially available, such as from Gesim and Brucker.

The MiniTrak device, for example, can transfer volumes as low as about 500 nL to a 1536 destination plate with contamination volumes (CV) between sample of less than 10%. The P10 (maximum volume=10 μl) tips used on the MiniTrak are disposable and can be washed out between runs, such as with ethanol, bleach or DMSO depending on the sensitivity of the sample transferred. The MiniTrak delivers sample directly to the bottom of each well.

In addition to piezo-dispensing tools, pin tools for delivery of small volumes also can be used. One such pin tool uses pins purchased from V&P Scientific demonstrably transfers as little as about 15 nL to each well of a 1536 destination plate with contamination volumes between sample of less than 10%. By dipping solid pins into a liquid and removing it, a uniform droplet of liquid hangs on the tip of the pin. This droplet is very uniform in volume. Its size is a function of several factors, including pin diameter, shape of the tip, surface tension on the pin, surface tension of the liquid, and the speed at which the pin is removed from the liquid. The pins can be washed with DMSO, methanol, and ethanol.

Destination plates can be kept indefinitely at −20 C or −80C. Storage of these destination plates allows for the assembly of an addressable and comprehensive collection of nucleic acids (“cDNA matrix”) that can be interrogated simultaneously and in toto in cell-based assays, such as those provided herein.

2. Reverse Transfection

Three microliters of serum free media containing an appropriate amount of a lipid-based transfection reagent, such as lipofectamine (Life Technologies), Fugene or other suitable agent, is deposited into each well in a multiwell plate, such as a plate containing 1536, 384 or other number of wells, using a multiwell liquid dispenser, such as one available from PerkinElmer or Cartesean Sinquad. The volume of the medium deposited is sufficient to cover the bottom of each well, thus allowing the nucleic acid sample to re-dissolve into the medium/reagent mixture regardless of variations in spotting of samples at the bottom of each well. The nucleic acid/reagent mixture is incubated for 15-45 minutes at room temperature. Target cells for transfection are detached (if necessary), and diluted to a concentration of 500,000-2,000,000 cells/ml (depending on cell type) in serum-containing medium. These cells are deposited into the nucleic acid/reagent-containing wells of plate, such as a 1536 chamber plate, with low volume dispensers (1-5 microliter) using a Cartesian Sinquad (above). Appropriate lids are applied, if needed, and the plate is transferred to a humidified tissue culture incubator, and the cells are assayed after 24-72 hours, or as appropriate.

3. Parallel High-throughput Viral Production

Viral production is accomplished when target cells described in #2 (above) are packaging/helper cells expressing viral packaging genes (i.e. gag, pol, env) in trans. Furthermore, arrayed nucleic acids (cDNA matrix) contain sequences required for viral packaging and subsequent expression in target cells. 2-4 days post-transfection of helper cells, supernatants are collected are transferred to a new plate (“viral destination plate”). Viral destination plates can be stored about −80° C. indefinitely, and can be collected to create a comprehensive and addressable viral cDNA matrices.

After the plates are thawed, target cells are infected by detachment and sebsequent addition to viral destination plates, which are placed in tissue culture incubators. Cells can be assayed after and appropriate time period.

An advantage of this technology is this increase in throughput over conventional transfections methods, permitting comprehensive studies of phenotype and pathways at the level of the genome. This is accomplished by the miniaturization and automation of the transfection procedure. By compartmentalizing each transfection into individual wells, futher processing, such as whole cell lysis (i.e. for luciferase), detection of secreted products, as well as viral production can be performed. Viral production will enable transduction of cell which are not highly transfectable, as well as facilitate the development expanded timeline assays which require long-term retention of transduced genes.

F. Modulation of Activity of Bioactive Small Molecules by Overexpression of cDNA Encoding Target Molecules

The activity of bioactive small molecules derived from screening with unknown molecular targets can be screened against a panel of known, relevant, over-expressed signaling pathway members and tested for modulation of the compound's effects. For exemplification of the methods herein, the NF-κB signal transduction pathway was interrogated with modulators of the activity thereof to identify the molecular targets of the modulators.

The NF-κB signal transduction pathway is induced by stimulation of the TNF or IL-1 (or other) lymphokine receptors, either by their respective ligands, by lipopolysaccharide (LPS), or by phorbol esters. This pathway, evolutionarily conserved in various forms across a wide range of species, is an essential component of the basic immune response in mammals. In mammals, activated NF-κB protein binds to the κ enhancer element, which controls expression of several genes involved in humoral immune response. Through a series of intracellular steps, the activation of the receptor promotes the phosphorylation and subsequent dissociation of the IκB inhibitor protein from the inactive NF-κB complex, allowing liberated NF-κB to translocate to the nucleus. Once active and inside the nucleus, NF-κB binds to the K enhancer element on the DNA and activates transcription of several apoptosis-related, cell growth-dependent, and B-cell-proliferative genes.

Using the methods provided herein for exemplification thereof, the TNF/NF-κB signaling pathway was interrogated by a panel of ˜1500 compounds of verified structure for inhibitors of NF-κB activation. Approximately twelve compounds had the desired effect without cytotoxic side-effects. Known TNF/NF-κB signaling genes were cloned into retroviral expression vectors and used in competition experiments with two of the compounds derived from screening. In these experiments, over-expression of NF-κB signaling pathway members was sufficient for induction of the NF-κB reporter gene, and could be specifically modulated by small molecule compounds derived from the cell-based screen. The experiments and results thereof are detailed in the Examples.

F. Modulation of Expression Using Oligonucleotides

Various genetic engineering and expression modification methods in which nucleic acid molecules are introduced into cells in a collection can be used to alter phenotypes in cells in the array. Such methods include chemical mutagenesis, transposon mutagenesis, antisense RNAi, dsRNAi, siRNA and transgene-mediated mis-expression.

Small oligonucleotides, such as RNA oligomers, including single and double-stranded RNA, are used to specifically target genes as a means of altering expression. A oligomer, such as an siRNA, that specifically targets, such as by degradation by an siRNA of a message, thereby reducing the level of endogenous protein encoded by that message. A plurality of such oligomers are designed and then arrayed such each locus in a collection, such an array, represents a single target. This plurality is introduced into cells to produce an addressable collection of cells, each containing a different oligomer. The cells are then scored for a phenotype.

For example, RNA interference (RNAi) (see, e.g. Chuang et al. (2000) Proc. Natl. Acad. Sci. U.S.A. 97:4985) can be employed. Interfering RNA (RNAi) fragments, particularly double-stranded (ds) RNAi, can be used to generate loss-of-function phenotypes, which can, in turn, be used, among other uses, to determine gene function. Methods relating to the use of RNAi to silence genes, in organisms including, mammals, C. elegans, Drosophila and plants, and humans are known (see, e.g., Fire et al. (1998) Nature 391:806-811 Fire (1999) Trends Genet. 15:358-363; Sharp (2001) Genes Dev. 15:485-490; Hammond, et al. (2001) Nature Rev. Genet. 2:110-1119; Tuschl (2001) Chem. Biochem. 2:239-245; Hamilton et al. (1999) Science 286:950-952; Hammond et al. (2000) Nature 404:293-296; Zamore et al. (2000) Cell 101:25-33; Bernstein et al. (2001) Nature 409: 363-366; Elbashir et al. (2001) Genes Dev. 15:188-200; Elbashir et al. (2001) Nature 411:494-498; International PCT application No. WO 01/29058; International PCT application No. WO 99/32619; International PCT application No. WO 01/36646). Double-stranded RNA (dsRNA)-expressing constructs are introduced into a host, such as an animal or plant using, a replicable vector that remains episomal or integrates into the genome. By selecting appropriate sequences, expression of dsRNA can interfere with accumulation of endogenous mRNA encoding a target protein.

Certain “antisense” fragments, i.e. that are reverse complements of portions of the coding sequence target polynucleotides can be used to alter phenotypes by inhibiting transcription or translation. The fragments are of lengths sufficient to alter expression and are generally at least 14 nucleotides in length, and typically contain 30, 50 up to about 150 nucleotides.

Alternatively, prior to, simultaneously with or subsequent to, the cells are exposed to a perturbation, and then the phenotypes of the resulting cells are scored. The perturbation can be one, for example that reverses the effect of the siRNA or an RNAi, thereby eliminating certain components in the pathway as targets or identifying possible targets or perturbations.

In all embodiments, the pattern of the resulting phenotypes is identified, and, associated with the oligomer and/or perturbation and is stored or recorded, such as in a database. Each result is an annotation for the nucleic acid molecule, such as the siRNA and target pair. The collection therefore is analyzed to identify those nucleic acid molecues, including, but are limited to, cDNA, DNA, siRNA, RNAi that perturb the pathway or perturbation and those that do not, thereby providing information regarding a molecular function and/or pathway.

G. Systems for Performing the Methods

The methods for identifying gene function are, in some embodiments, conducted using a high throughput processing system such as those described in International Patent Application PCT/US01/32454, which was filed on Oct. 15, 2001. Typically, these systems include a plurality of work perimeters and a plurality of rotational robots, e.g., about 2 to about 10 robots. Each rotational robot is typically associated with one or more member of the plurality of work perimeters. For example, the robots each have a reach which reach defines the work perimeter associated with that robot. The plurality of work perimeters and the plurality of rotational robots are configured to allow transport of one or more sample holder (such as a microtiter plate) along a multi-directional path, e.g., to provide a flexible transport system for a plurality of sample holders. In addition, the systems comprise at least one device associated with each work perimeter. Typically, at least one of the work perimeters has two or more devices exclusively within the reach of the associated rotational robot for that work perimeter. The system is configured to provide non-sequential transport between the two or more devices, with each device being accessible by at least one of the rotational robots. To further aid the transport of the plurality of sample holders, the systems typically comprise one or more transfer station associated with at least a first work perimeter and a second work perimeter. The transfer stations provide transportation of samples (either by transferring the holders themselves or by transferring aliquots of samples from one sample holder to another) between work perimeters, e.g., from the first work perimeter to the second work perimeter.

In some embodiments, the methods for identifying gene function are conducted using a gripper that is configured to hold and precisely position microtiter plates. The gripper mechanism is typically configured to hold the various size multiwell plates, e.g., including, but not limited to 1536-well plates. Gripper mechanisms are described, for example in U.S. application Ser. No. 09/793,254, entitled “Gripper Mechanism,” filed Feb. 26, 2001, and in International Patent Application No. ______, entitled “GRIPPING MECHANISMS, APPARATUS, AND METHODS,” which was filed on Feb. 26, 2002 as Attorney Docket No. 36-000410PC, which provides gripper apparatus, grasping mechanisms, and related methods for accurately grasping and manipulating objects with higher throughput than preexisting technologies. In certain embodiments, for example, grasping mechanisms are resiliently coupled to other gripper apparatus components. In other embodiments, grasping mechanism arms include support surfaces and height adjusting surfaces to determine x-axis and z-axis positions of objects being grasped. In certain other embodiments, grasping mechanism arms include pivot members that align with objects as they are grasped. In some of these embodiments, pivot members include the support surfaces and height adjusting surfaces. In other embodiments, the arms of grasping mechanisms include stops that determine y-axis positions of objects that are grasped. Essentially any combination of these and other embodiments described herein is optionally utilized together.

To reduce contamination and evaporative effects, it is sometimes desirable to provide at least some of the sample holders with lids. A lid that sufficiently seals a sample holder not only reduces evaporation and contamination, but allows gases to diffuse into sample wells more consistently and reliably. Lids generally have a gripping structure, such as a gripping edge, that a robotic arm gripper can engage. Accordingly, a robot is able to lid and delid the specimen plate as needed. Suitable specimen plate lids are described in PCT/US01/15366, entitled “Specimen Plate Lid and Method of Using”, filed May 10, 2001, which discloses specimen plate lids for robotic use, and is incorporated herein by reference as if set forth in its entirety. In one embodiment, the lids comprise a cover having a top surface, a bottom surface, and a side. An alignment protrusion extends from the side of the cover and is positioned to cooperate with an alignment member of a multiwell plate. The alignment protrusion does not frictionally mate with sidewalls of the specimen plate when the lid is placed on the specimen plate, therefore allowing the lid to be removed from the plate without disturbing the plate. The lids typically have a sealing perimeter positioned on the bottom surface of the cover. The alignment protrusion facilitates aligning the lid to the plate so that a seal is compressibly received between the sealing perimeter and a sealing surface of the multiwell plate. The lids are of sufficient weight to compress the seal and form a tight seal between the lid and the plate. For example, the lids typically weigh between about 100 grams and about 500 grams. Stainless steel is one example of a suitable material for the lids. A lidding and/or de-lidding station is also optionally included as a device in the present systems, e.g., to add and/or remove the lids described above to or from the sample holders. Alternatively, the entire robotic system is optionally enclosed, thus creating a controlled environment, to further reduce contamination and evaporative effects.

In some embodiments, the methods for identifying gene function are performed using one or more automated systems for precisely positioning an object, as described in PCT/US01/19274, entitled “Automated Precision Object Holder and Method of Using Same,” which was filed Jun. 15, 2001 and in U.S. patent application Ser. No. 09/929,985, filed Aug. 14, 2001. Microtiter plates must be placed precisely under liquid dispensers to enable a liquid dispenser, for example, to deposit samples or reagents into the correct sample wells. A tolerance of about 1 mm, which can sometimes be obtained by systems that do not include this type of automated precision object holder, is adequate for some low density microtiter plates. However, such a tolerance is often unacceptable for high density plates, such as a plate with 1536 wells. Indeed, a positioning error of one mm for a 1536 well microtiter plate could cause a sample or reagent to be deposited entirely in the wrong well, or cause damage to the system, such as to needles or tips of the liquid dispenser. Accordingly, positioning devices as described in U.S. application Ser. No. 09/929,985 and International PCT application No. PCT/US01/19274 are optionally used in the methods herein, particularly when 1536 well plates are used.

These positioning devices have at least a first alignment member that is positioned to contact an inner wall of the microtiter plate when the microtiter plate is in a desired position on the support. An inner wall 88 of a microtiter plate is shown in, for example, FIG. 13 of PCT/US01/19274. In some embodiments, two or more alignment members are positioned to contact a single inner wall of the microtiter plate when the microtiter plate is in the desired position on the support. The use of an inner wall of the microtiter plate as an alignment surface greatly increases the precision with which the microtiter plate is positioned on the support compared to, for example, aligning the microtiter plate using an outer wall, thereby facilitating further processing of the samples contained in the microtiter plate. The positioning devices can further include at least a second alignment member that is positioned to contact a second wall of the microtiter plate when the microtiter plate is in the desired position on the support. This second wall is preferably an inner wall of the microtiter plate. The positioning devices can include: a) a first pusher for moving the plate in a first direction so that a first alignment surface of the object contacts a first set of one or more alignment members; and b) a second pusher for moving the plate in a second direction so that a second alignment surface of the object contacts a second set of one or more alignment members. In presently preferred embodiments, either or both of the pushers includes a lever pivoting about a pivot point. The lever can be operably attached to a spring or equivalent, which causes the pusher to apply a constant force to the object to, for example, move the object in the first direction against the first set of alignment members. The positioner in operation, including the use of alignment tabs 30, is illustrated in the copending application (see, U.S. application Ser. No. 09/929,985).

The automated precision object holders can also include a retaining device for retaining a microtiter plate in a desired position on a support. These retaining devices can include, for example, a vacuum plate which, when a vacuum is applied, holds the microtiter plate in the desired position. The vacuum plate, in some embodiments, has an interior surface and a lip surface, with the interior surface being recessed relative to the lip surface.

The methods herein can be perfromed in microtiter plates, which are optionally encoded with a symbology, such as a bar code. The microtiter plates generally those that have 300 or more wells. Such methods can be automated and can employ a positioning device that inlcudest least a first alignment member that is positioned to contact an inner wall of the microtiter plate when the microtiter plate is in a desired position on a support. The positioning device can further include a pusher that can move a microtiter plate in a first direction to bring the inner wall of the microtiter plate into contact with one or more of the alignment members.

The microtiter plates also can be covered with a lid. Such lids can include a cover having a top surface, a bottom surface, and a side; an alignment protrusion extending from the side of the cover, the alignment protrusion positioned to cooperate with an alignment member of the microtiter plate, such that the alignment protrusion does not frictionally mate with sidewalls of the microtiter plate when the lid is placed on the microtiter plate; and a sealing perimeter positioned on the bottom surface of the cover. The alignment protrusion facilitates aligning the lid to the plate so that a seal is compressibly received between the sealing perimeter and a sealing surface of the microtiter plate when the lid is placed on the microtiter plate. Such lids can be stainless steel.

In performing the methods, the microtiter plate can be manipulated using a robotic gripper that includes one or more components selected from among:

a. moveably coupled arms that are structured to grasp the microtiter plate, wherein at least one arm comprises a stop, and wherein at least two grasping mechanism components are resiliently coupled to each other by a resilient coupling;

b. moveably coupled arms that are structured to grasp the microtiter plate, wherein at least one arm comprises at least one support surface to support the microtiter plate and at least one height adjusting surface that pushes the microtiter plate into contact with the support surface when the arms grasp the microtiter plate; and

c. moveably coupled arms that are structured to grasp the microtiter plate, wherein at least one arm comprises a pivot member that aligns with the microtiter plate when the arms grasp the microtiter plate.

H. Automation

The steps of the methods can be automated or partially automated in any combination with manual steps. Operator input, as appropriate, can precede, follow or intervene between the steps, if desired. Software or hardware that includes computer readable instructions for implementing the automated steps also can be included in the systems and programs. An operator can interface with the computer to control automation, the steps automated, and repetition of any step.

For example, a microscope used to detect a fluoresecent signal or bioluminescence can be automated with a computer-controlled stage to automatically scan the entire array. Similarly, the microscope can be equipped with a phototransducer, such as photomultiplier, a solid state array, a CCD camera and other imaging devices, attached to an automated data acquisition system to automatically record the fluorescence signal produced by hybridization. Such automated systems are known (see, e.g., U.S. Pat. No. 5,143,854).

The microscope can be operatively connected to a data acquisition system for recording and subsequent processing of the fluorescence or other electromagnetic radiation output intensity information and calculating the absolute or relative amounts of gene expression. Following calculation of relative values, cells with nucleic acid introduced therein whose output has changed are identified. The nucleic acid and/or encoded product is a candidate target for the effector of the change. Thus, the entire process or any part of the process from the initial identification of modulators to designing primers appropriate for cloning a gene regulatory region to preparation of the cells to identification of outputs from the collection of cells can be automated.

Thus, methods can be performed in a high throughput processing system. Such systems can include one or more of:

a. a plurality of rotational robots, wherein each of the rotational robots has a reach which defines a work perimeter associated with that rotational robot;

b. at least one device associated with each of the work perimeters, wherein at least one of the work perimeters has two or more devices exclusively within the reach of the rotational robot associated with that work perimeter;

c. one or more transfer stations associated with at least a first work perimeter and a second work perimeter, for transferring one or more samples from the first work perimeter to the second work perimeter; and

d. a plurality of microtiter plates, which microtiter plates are transported between two or more devices or between two or more work perimeters during operation of the system.

The following examples are included for illustrative purposes only and are not intended to limit the scope of the invention. The specific methods exemplified can be practiced with other species. The examples are intended to exemplify generic proceses.

### EXAMPLE 1

Construction of Reporter Cell Lines

cDNA Library Preparation

cDNA libraries were generated using Life Technologies Superscript Plasmid System and standard procedures. The cDNA for each library was produced from Clontech poly-A+mRNA from the selected tissue source. First strand synthesis was primed using docking primers with a NotI site. The results of first and second strand synthesis were tracked by incorporation of a small amount of α-³²P dGTP into the reactions.

Syntheses were analyzed for fidelity by alkaline gel electrophoresis and for percent incorporation by chromatography (Whatman GF/C Filters). Sal I adaptors were ligated to the cDNA fragments and subsequently cleaved with Not I. Size fractionation of the cDNA was performed using columns provided in the Superscript Plasmid System Kit. Fractionated cDNA was then purified and ligated into precut Not I-Sal I pSPORT-1 (or the desired vector). Ligated cDNA was electroporated into ElectroMAX DH10B electrocompetent cells (Life Technologies) and plated on selective media to determine the titer. The remaining electroporated cells were frozen in glycerol for future use.

Normalization of cDNA Libraries Through Cold Colony Picking

Frozen aliquots of previously generated cDNA libraries were thawed and amplified once in 100 ml cultures. Each 100 ml culture was frozen in glycerol in 1 ml aliquots. An aliquot was then titered to determine the number of colony forming units/ml. Large bioassay trays (245 cm×245 cm) were innoculated with the library and grown overnight. Colonies were picked with a Genetix Q-Pix robot at a rate of 4200-4600 colonies/hr into selective media containing 8% glycerol. Colonies were grown and then frozen as stock plates. Stock plates were thawed and used to innoculate fresh 384-well plates. These plates were then used as source plates to grid colonies using a Genetix Q-box onto Hybond-N membranes (Amersham). Colonies were grown overnight and the membranes processed using alkaline lysis and UV crosslinking to generate plasmid DNA representing each individual colony. Membranes were then hybridized to labeled cDNA representing the source tissues. A four-to ten-fold reduction in redundancy of the cDNA clones was achieved by this method.

Normalization Through Directed Open Reading Frame (ORF) Amplification

Primers were designed against all known proteins such that the amplification product contained the start methionine and the entire ORF through the stop codon followed by a Pac1 site. These primers were used in the PCR against mRNA samples where the desired target was present at (average difference level) AD>200 by Genechip (Affymetrix) analysis using Pfu Turbo (Stratagene). PCR products were isolated by agarose gel electrophoresis, digested in the gel, and ligated into a Pac1/EcoRV adapted pENTR derivative (Gibco-BRL). These entry vector clones were transferred via the Gateway recombination system into the desired retroviral or transient transfection vector.

Plasmid pNFκB-Luc

The plasmid pNFκB-Luc (available from Clontech, see, SEQ ID No. 3), which was designed for monitoring the activation of NFKB signal transduction pathway ((1998) CLONTECHniques XIII(3):24-25; Baeuerle et al. (1996) Cell 87:13-20; Baeuerle (1998) Curr. Biol. 8:R19-R20; Peltz (1997) Curr Opin. Biotechnol. 8:467-473), contains the firefly luciferase (luc) gene from Photinus pyralis (De Wet et al. (1987) Mol. Cell Biol. 7:725-737; see, e.g., International PCT Application No. WO 95/25798, which provides Photinus luciferase in which the glutamate at position 354 is replaced lysine). This vector contains four tandem copies of the NFκB consensus sequence fused to a TATA-like promoter (PTAL) region from the Herpes simplex virus thymidine kinase (HSV-TK) promoter. NF-κB binds to the κB4 element on the vector and initiates transcription of luciferase. After endogenous NFκB proteins bind to the kappa (κ) enhancer element (κB4), transcription of the pNFκB-luc is induced and the reporter gene, luciferase, is activated. The luciferase coding sequence is followed by the SV40 late polyadenylation signal to ensure proper, efficient processing of the luc transcript in eukaryotic cells. Located upstream of NFκB is a synthetic transcription blocker (TB), which is composed of adjacent polyadenylation and transcription pause sites for reducing background transcription (Eggermont et al., (1993) EMBO J. 12:2539-2548). The vector backbone also contains an f1 origin for single-stranded DNA production, a pUC origin of replication, and an ampicillin resistance gene for propagation and selection in E. coli.

The vectors are available from Clontech in three forms: pNF-κB-Luc contains the firefly luciferase gene; PNF-κB-SEAP contains the secreted alkaline phosphatase (SEAP) gene; and pNF-κB-d2EGF contains the gene encoding destabilized enhanced green fluorescent protein. After transfection of the reporter vector into an appropriate cell line, the NF-κB pathway can be activated using various stimuli. Induction of the pathway permits endogenous NF-κB to bind to the four tandem copies of the kappa enhancer element (κB4) located upstream of the reporter gene on the vector. Binding of NF-κB enhances the association of the cells' general transcription machinery with the herpes simplex virus thymidine kinase (HSV-TK) promoter fused downstream of B4, resulting in high induction levels of reporter gene transcription.

Reporter Vector Construction

A 1912 bp region from the pNFκB-Luc Mercury Signal Transduction Vector (Clontech; see SEQ ID No. 3) containing the four tandem copies of the NF-κB consensus sequence fused to a TATA-like promoter (P_{TAL}) region from the Herpes simplex thymidine kinase (HSV-TK) promoter followed by the luciferase coding sequence was amplified. The sequences of the PCR primers were:

SEQ ID No. 1

5′-GGCCTAGTCCTCGAGGGGAATTTCCGGGAATT-3′ and

SEQ ID No. 2

5′-GGCCTAGTCGGATCCTTACACGGCGATCTTT-3′.

The amplified region was cloned into the Xho1 and BamH1 sites of the a SIN retroviral reporter vector, which contains the neomycin resistance gene for G418 selection. The resulting vector was designated SKBL-N.

Stable Reporter Cell Generation:

Day 1: HEK293 cells were seeded at 8×10⁵ cells/well in six-well plates.

Day 2: For virus production, HEK293 cells in the six-well plate were transiently transfected with a cocktail of 2.5 μg reporter vector (SKBL-N) and retroviral packaging plasmids; 2.5 μg Gag-Pol vector and 2.5 μg VSV-G expression vector using CalPhos Mammalian Transfection Kit (Clontech). Transfections were done in the presence of 50 μM chloroquine. The transfection medium was replaced with fresh growth medium six to eight hours after transfection.

Day 3: 24 hours after transfection, the medium containing retroviral vector was collected and replaced with fresh medium for either HEK293 cells or Jurkat T cells. Separately, 8×10⁵ HEK293 cells were seeded in a six-well plate or 1×10⁶ Jurkat cells in 3 mL media.

Day 4: Retroviral supernatants from the transfected HEK293 cells were harvested, filtered through μm filter, and used to infect the HEK293 cells and Jurkat T cells in the presence of 5 μg/ml protamine sulfate.

Day 5: The transduced cells were changed into fresh medium 16 hours after transduction.

Day 6: The transduced HEK293 and Jurkat cells were transferred to 10 cm dishes and selected (for SKBL-N) in geneticin (50 mg/ml, Gibco BRL) at a final concentration of 800 ug/ml. The cells were maintained in G418 for a minimum of four to five days and then assayed.

Day 7: HEK293 and Jurkat NF-κB reporter cells were plated and treated with a dose-response of human TNF-alpha for 2 to 24 hours, lysed and treated with Bright-Glo luciferase reagent (Promega) and luminescence measured with the LJL Acquest luminometer.

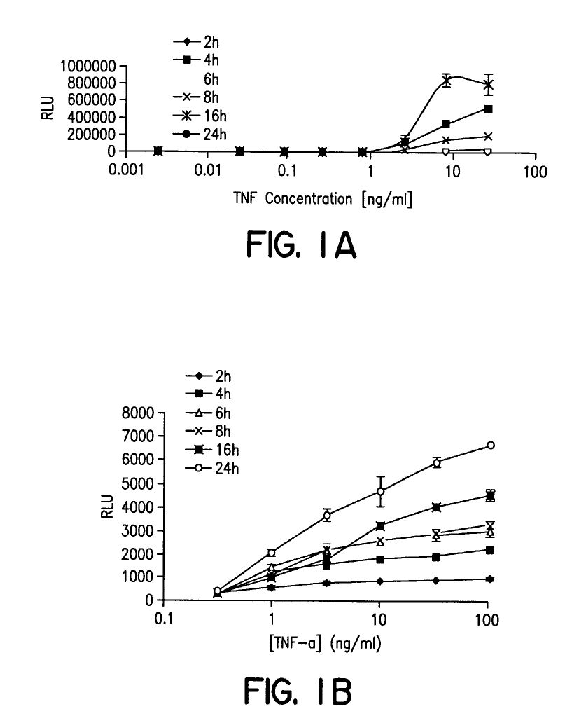

Both NF-κB reporter cell lines were inducible with TNF-alpha, as demonstrated by the time-course, dose-response experiments shown in FIG. 1.

### EXAMPLE 2

In Cellulo Competition

HEK293T NF-κB reporter cells were seeded at 7000 cells/well in 384-well plates in triplicate. Eighteen hours later, cells were treated with TNF-alpha (10 ng/mL) in the absence of or presence of 1, 2 or 5 mM sodium salicylate. Cells in other wells were transfected with mammalian expression vectors encoding wild-type human IKK-beta (50 ng) or NF-κB p65 (10 ng) by calcium phosphate. Eight hours post transfection, fresh medium was added with 1, 2 or 5 mM sodium salicylate and incubated for an additional 16 hours. At 24 hours post TNF addition or post transfection, cells were lysed and incubated with Bright-Glo (Promega) and Relative Light Units (RLUs) were determined using the LJL Acquest luminometer.

Alternatively, Jurkat NF-κB reporter cells were seeded at 30,000 cells per/ml in 384-well microtiter plates. Recombinant retroviruses encoding wild-type IKK-beta or NF-κB p65 were generated and used to transduce Jurkat reporter cells. Untransduced cells were treated with 1, 2 or 5 mM salicylate for 30 minutes prior to stimulation with TNF-alpha (10 ng/ml). For transduced cells, 4 hours post retroviral incubation, cells were treated with either 0, 1, 2 or 5 mM salicylic acid. In either case, 16 hours post stimulus addition, cells were were lysed and incubated with Bright-Glo (Promega) and Relative Light Units (RLUs) were determined using the LJL Acquest luminometer. Results are shown in FIG. 2.

In both sets of experiments, over-expression of IKK-beta, but not NF-κB p65, could titrate out the effect of salicyclic acid on induction of the NF-κB reporter, demonstrating that salicylic acid acts upstream of p65 activation on the IKK-beta kinase subunit.

### EXAMPLE 3

Modulation of Activity of Bioactive Small Molecules by Overexpression of cDNA Encoding Target Molecules

The activity of bioactive small molecules derived from screening with unknown molecular targets can be screened against a panel of known, relevant, over-expressed signaling pathway members and tested for modulation of the compound's effects.

Cell Plating: Jurkat NF-κB reporter cells were seeded at 5 μL per well in Greiner 1536-well micro-plates using the Cartesian synQUAD. Settings for the 24,000 step motor were such that a 100 μL syringe would provide a volume per step of 4.2 nL; timing was controlled by a master dispenser solenoids and stepper motors which moved the stage and controlled the syringe pumps. The result was extremely rapid “on-the-fly” dispensing, similar to an inkjet printer. This synchronicity also allowed modulation of the volume of each drop (with the syringe speed and solenoid open time), as well as the placement of the drops (by varying the table speed and syringe speed).

Compound addition: Four Falcon 384-well plates containing compounds (Aldrich) dissolved in dimethylsulfoxide (DMSO) to a final concentration of 1 mM (excepting the last two columns which were just DMSO) were transferred to the Jurkat reporter cells. The operation was semi-automated using a Robbins Hydra-384 with 100 μL DuraFlex needles for precision dispensing. A total of 50 nl of each compound was transferred to the 5 ul of cells, resulting in a dilution to 1% DMSO and 10 uM compound. Plate positioning was controlled with a modified Wizard protocol in order to transfer accurately source fluid to destination plates in the four designated “quadrants” of the 1536-well cell plate. Coefficients of variation (CVs) before and after compound addition was determined by transferring liquid from a FITC solution bath to 384 well plates and read on the LJL Acquest in fluorescence modality.

Stimulus and detection addition: After 30 minute incubation with compounds, a solution of TNF-alpha (Sigma) diluted to 60 ng/ml and transferred to cells using the Cartesian synQUAD such that 1 μL of stimulus was added per well to a final dilution of approximately 10 ng/ml final. Cells were incubated for 16 hours at 37° C., 5% CO₂ in a Forma humidified incubator, then returned to the Cartesian for addition of 1 μL of a 7× solution of Alamar Blue (Trek Diagnostics, fluorescent indicator of cell viability/proliferation). Cells were incubated with the Alamar Blue for three hours then read on the LJL Acquest in fluorescence mode at 100,000 us/well. Next, the cell plate was assayed for luciferase activity by addition of 5 μL/well Bright-Glo (Promega). Precisely five minutes after addition of Bright-Glo, the cell plate was read in luminescence mode in the LJL Acquest at 100,000 us per well. Wells treated with compounds in which fluorescent signals were >90% of the mean across the plate, and below 50% of the mean across the plate for luminescence were identified and compounds hit picked for future studies. The twelve compounds picked for follow-up were tested for IC50 values, using half-log dilutions of each (ranging from 100 uM to 10 nM). IC50 values were also determined in the HEK293 NF-κB reporter gene assay in the same manner.

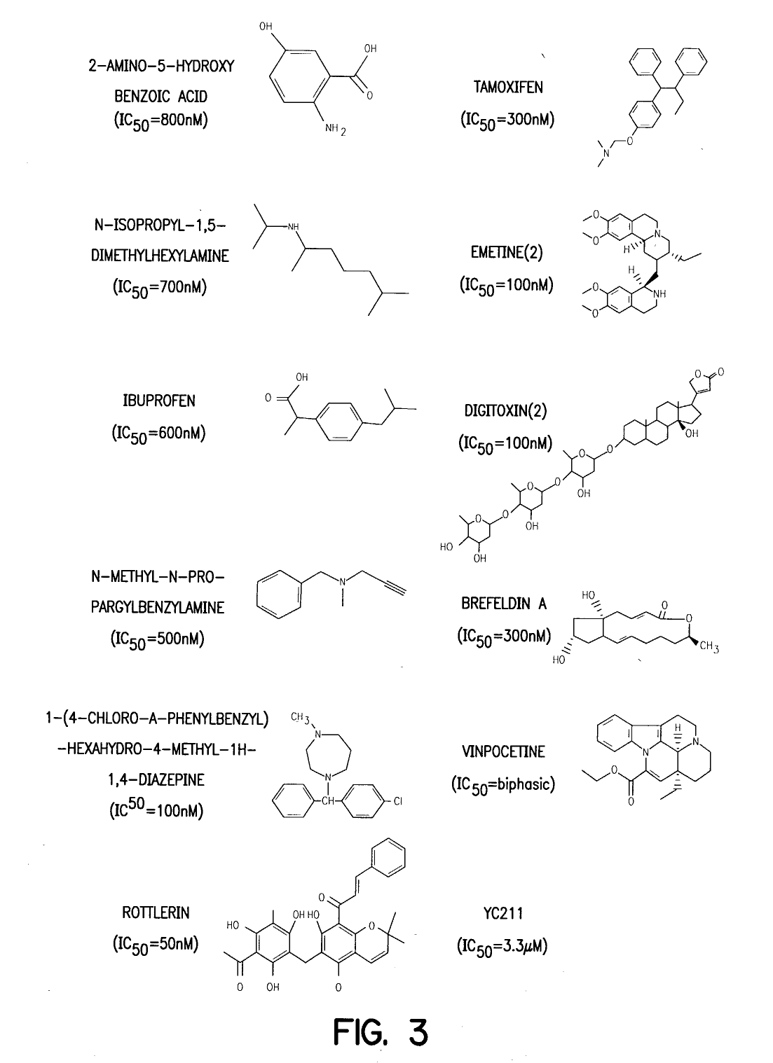

FIG. 3 shows twelve compounds that were isolated by high-density cell-based screening as described above. Each compound was capable of blocking TNF-induced NF-κB activity as assessed by an NF-κB dependent reporter cell assay. The name and compound structure is shown together with the IC50 value for each compound.

### EXAMPLE 4

In Cellulo Competition Assay

cDNA library construction: A fetal liver/brain tissue cDNA library was purchased from Clontech and transferred into the retroviral expression vector ViP3 or MSCV-iN by standard molecular biology techniques. Bacterial colonies transformed by the library constructs were plated and picked using the Q-Pix (Genetix) into 96-well plates. Approximately 2000 colonies were picked and grown in LB-ampicillin media in 96-well cartridges overnight followed by DNA miniprep using the Qiagen 9600. DNA yields for several clones from each plate were determined by spectrophotometry. Fifty microliters of DNA solution for every 4 96-well plates was transferred to individual wells of a 384-well Falcon plate and stored at −20° C. The two right hand columns of every 384-well plate were left empty for controls.

TNF pathway member cloning: Primers specific for TNFR(p55), TRAF2, NIK, IKK-beta, IKK-alpha and NF-κB p65 were ordered and used to PCR amplify these genes from the fetal liver/brain cDNA library. Full-length genes were amplified, isolated and cloned into the retroviral vector termed ViP3. Sequences were verified by Sanger dideoxy termination reaction/ABI prism sequencing. 100 ng/ml of each cloned TNF pathway member was placed in an empty well in the 384-well Falcon plates containing random cDna library members.

Screening and Small Complementation: HEK293 NF-κB reporter cells were plated at 7000 cells/well in 384-well Greiner clear bottom plates using a Titertek Multidrop. Cells were incubated for 8 hours before transfection of the cDNA libraries. Cells were treated with either Rottlerin, YC211 or control DMSO (1% final) using the Hydra-384. Thirty minutes compound treatment, The Hydra was used again to mix two μL DNA with 8 ul of a premixed solution 61 μl 2m CaC12, 440 μH20 distributed into a 384-well intermediate plate. Then, 10 ul of a 2× Hepes Buffered Saline solution (HBS, pH 7.0) was mixed with the DNA and pipetted automatically for 5 seconds followed by 10 μL addition of the transfection solution to HEK293 NF-κB reporter cells. After transfected plates of cells were incubated at 37° C. for 16 hours, Bright-Glo was added to each well using a twelve-head multi-channel pipettor, incubated for five minutes then read on the LJL Acquest in luminescence mode. Controls used in this experiment were limited to p65, IKK-beta, NIK and IKK-alpha. Additionally, retroviral vectors encoding firefly luciferase alone were plated in 384-wells and transfected into wild-type HEK293 cells to determine transfection efficiency and CVs.

cDNA Modulation of the Effects of Bioactive Small Molecules:

HEK293 NF-κB reporter cells were plated in 96-well plates at 28,000 cells/well in D'MEM media containing 10% FBS, pen-strep antibiotics and 1 mM glutamine. Sixteen hours after seeding, cells were treated with Rotlerrin, YC211 at their IC50 concentrations (50 nM, 3.3 μM, respectively) or DMSO before transfection with 100 ng/ml TNFR, TRAF2, NIK, IKK-beta, IKK-alpha, p65 expression vectors or stimulated with 5 ng/ml TNF-alpha. After 24 hours, samples were treated with Bright-Glo and analyzed using the LJL Acquest luminometer.

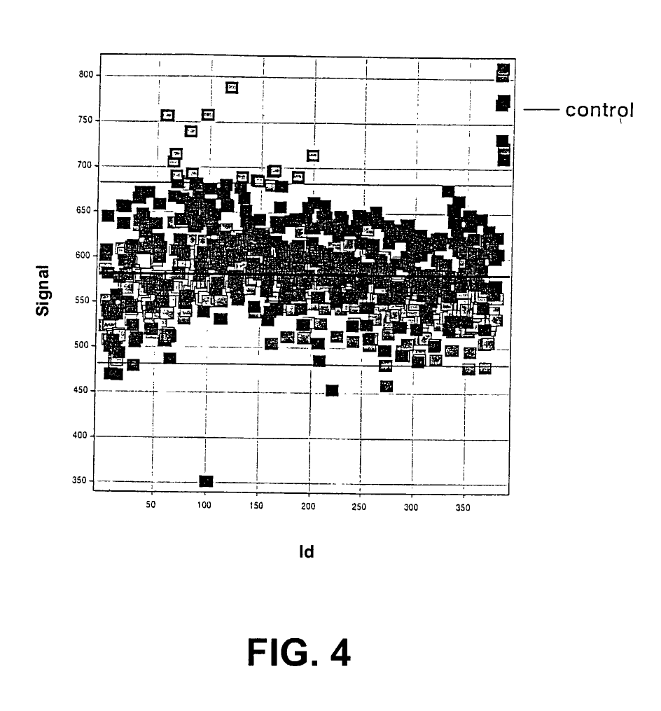

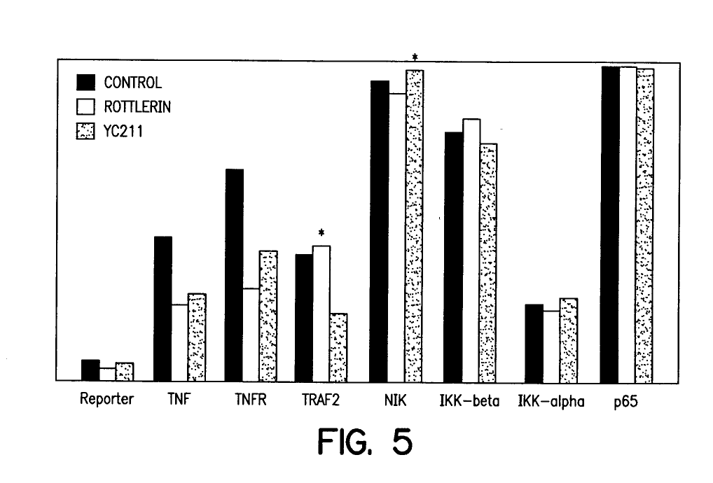

The results are shown in FIGS. 4 and 5. FIG. 4 is a scatter plot of the results obtained from two of the 384-well plates treated with 1% DMSO control only and no inhibitor compound and shows the activity of the cDNA overexpressed in the HEK293 NF-KB cell line for each cDNA. As evidenced by the positive signals shown to the right, where the control wells reside), each of the four controls (IKK-beta, p65, IKK-alpha and NIK were positive. Several of the random library members also resulted in increased luminescence. The plates treated with Rottlerin and YC211 gave similar results. This demonstrates that cDNA library screens in arrayed formats can be performed using industrial laboratory automation to identify true pathway signaling effectors.

FIG. 5 shows the effects of specific cDNA overexpression on the effects of bioactive small molecules in a cellular reporter gene assay. These cells are HEK293 NF-κB-luciferase reporter cells. The stimulus or reagent introduced is shown on the x-axis. The y-axis shows the relative luciferase activity induced by each stimulus. The stars represent areas of interest. For example, Rottlerin is able to block signals induced by TNF, TNFR, but not TRAF2, suggesting that the target for Rottlerin is downstream of TNFR but upstream of TRAF2. Alternatively, TNF, TNFR, TRAF2, but not NIK overcome the inhibition of YC211, indicating that the target of NIK acts downstream of TRAF2 and upstream of NIK.

Since modifications will be apparent to those of skill in this art, it is intended that this invention be limited only by the scope of the appended claims.

What is claimed is:

1. A method for identifying a function of endogenous gene by modulating the level of a product encoded by the endogenous gene, the method comprising:
a) introducing nucleic acid molecules into populations of reporter cells to form an addressable collection of cell populations, wherein cells of a first cell population comprise a different introduced nucleic acid from cells of at least a second cell population and
b) identifying cell populations in the collection in which cells exhibit a phenotype that is different in the presence of the introduced nucleic acid molecule from the phenotype exhibited in its absence, thereby identifying a nucleic acid molecule that modulates the level of a product of an endogenous gene or genes that effect the phenotype and identifying the function of the endogenous gene or genes.

2. The method of claim 1, wherein the nucleic acid molecule introduced into each cell population comprises a known polynucleotide sequence.

3. The method of claim 1, wherein the addressable collection comprises at least 1000 cell populations, each of which comprises a different introduced nucleic acid molecule.

4. The method of claim 3, wherein the addressable collection comprises at least 10,000 cell populations.

5. The method of claim 1, wherein the introduced nucleic acid molecules represent a portion of a transcriptome derived from a cell, tissue, organ, organism or that comprises a pathway.

6. The method of claim 5, wherein the introduced nucleic acid molecules represent at least 50% of transcribed nucleic acids in a genome or transcriptome of a cell.

7. The method of claim 6, wherein the introduced nucleic acid molecules represent at least 75% of transcribed nucleic acids that comprise a genome or transcriptome of a cell.

8. The method of claim 5, wherein the introduced nucleic acid molecules comprise a transcriptome that contains the transcripts from a genome or cDNA molecules derived from the transcripts from a genome.

9. The method of claim 1, wherein the introduced nucleic acid comprises nucleic acid that encodes members of a targeted pathway.

10. The method of claim 1, wherein each of the cell populations is not in fluid contact with other cell populations.

11. The method of claim 10, wherein each set cell population of the addressable collection is in a well of a microwell plate.

12. The method of claim 11, wherein the density of wells in the micro-well plate is 300 wells/plate or greater.

13. The method of claim 12, wherein the density of wells in the micro-well plate is 1500 wells/plate or greater.

14. The method of claim 1, further comprising:
c) recording data representative of the change in phenotype of the identified cells and the corresponding introduced nucleic acid molecules.

15. The method of claim 14, wherein the data is recorded in a database.

16. The method of claim 1 that is automated.

17. The method of claim 1, wherein the introduced nucleic acid molecule decreases the level of the product of the endogenous gene.

18. The method of claim 17, wherein the introduced nucleic acid molecule is interfering RNA (RNAi) or is siRNA.

19. The method of claim 17, wherein the introduced nucleic acid is a DNA molecule that is transcribed to yield an RNAi or an siRNA.

20. The method of claim 17, wherein the introduced nucleic acid molecule comprises antisense oligonucleotides.

21. The method of claim 1, wherein the introduced nucleic acid molecule is DNA.

22. The method of claim 1, wherein the introduced nucleic acid molecule increases the level of the product of the endogenous gene.

23. The method of claim 22, wherein the product of the endogenous gene is an mRNA that encodes a polypeptide in a targeted pathway.

24. The method of claim 1, wherein the introduced nucleic acid molecule is a cDNA that encodes a protein.

25. The method of claim 1, wherein the introduced nucleic acid molecule decreases the level of an endogenous mRNA.

26. A method for identifying the targets of a perturbagen by modulating the level of an endogenous messenger RNA, comprising:
a) introducing a nucleic acid molecule into populations of reporter cells to form an addressable collection of cell populations, wherein cells of a first cell population comprise a different introduced nucleic acid from cells of at least a second cell population; and
b) exposing the cells to a perturbagen that potentially alters a phenotype; and
c) identifying cell populations in the collection in which cells exhibit a phenotype that is different in the presence of the introduced nucleic acid molecule and the perturbagen compared to the phenotype exhibited by the cells in the absence of the introduced nucleic acid molecule and the perturbagen;
wherein a) and b) are performed either simultaneously or sequentially in either order, and the method thereby identifies a target or targets of the perturbagen.

27. The method of claim 26, wherein the introduced nucleic acid encodes a potential target of the perturbagen.

28. The method of claim 26, wherein the addressable collection comprises at least 1000 cell populations, each of which comprises a different introduced nucleic acid molecule.

29. The method of claim 28, wherein the addressable collection comprises at least 10,000 cell populations.

30. The method of claim 26, wherein the introduced nucleic acid molecules represent a portion of a transcriptome derived from a cell, tissue, organ, organism or that comprises a pathway.

31. The method of claim 30, wherein the introduced nucleic acid molecules represent at least 50% of transcribed nucleic acids in a genome or transcriptome of a cell.

32. The method of claim 31, wherein the introduced nucleic acid molecules represent at least 75% of transcribed nucleic acids that comprise a genome or transcriptome of a cell.

33. The method of claim 30, wherein the introduced nucleic acid molecules comprise a transcriptome that contains the transcripts from a genome or cDNA molecules derived from the transcripts from a genome.

34. The method of claim 26, wherein the introduced nucleic acid comprises nucleic acid that encodes members of a targeted pathway.

35. The method of claim 26, wherein each of the cell populations is not in fluid contact with other cell populations.

36. The method of claim 35, wherein each cell population of the addressable collection is in a well of micro-well plate and cells that contain each introduced nucleic acid are present in a different well from cells that contain other introduced nucleic acids.

37. The method of claim 36, wherein the density of wells in the micro-well plate is 300 wells/plate or greater.

38. The method of claim 37, wherein the density of wells in the micro-well plate is 1500 wells/plate or greater.

39. The method of claim 26 that is automated.

40. The method of claim 26, further comprising:
c) recording data representative of the change in phenotype of the identified cells and the corresponding introduced nucleic acid molecules and perturbagens.

41. The method of claim 40, wherein the data is recorded in a database.

42. The method of claim 26, wherein the introduced nucleic acid molecule decreases expression of the product of the endogenous gene.

43. The method of claim 42, wherein the introduced nucleic acid molecule is interfering RNA (RNAi) or is siRNA.

44. The method of claim 42, wherein the introduced nucleic acid is a DNA molecule that is transcribed to yield an RNAi or an siRNA.

45. The method of claim 26, wherein the introduced nucleic acid is DNA.

46. The method of claim 26, wherein the introduced nucleic acid increases the level of the product of the endogenous gene.

47. The method of claim 46, wherein the product of the endogenous gene is an mRNA that encodes a polypeptide in a targeted pathway.

48. The method of claim 26, wherein the introduced nucleic acid is cDNA that encodes a protein.

49. The method of claim 26, wherein the introduced nucleic acid decreases the level of an endogenous mRNA.

50. The method of claim 26, wherein the perturbagen comprises a compound or condition that is an antagonist of expression of a gene or a cellular activity.

51. The method of claim 50, wherein prior to exposure to the antagonist, the cells are exposed to an agonist of expression of the gene.

52. The method of claim 26, wherein the perturbagen is a compound.

53. The method of claim 52, wherein the compound is a nucleic acid molecule.

54. The method of claim 52, wherein the compound is a small molecule effector compound.

55. The method of claim 26, wherein the perturbagen is an agonist of expression of a gene or a cellular activity.

56. The method of claim 1, wherein the reporter cells comprise a regulatory region operatively linked to nucleic acid encoding a reporter protein.

57. The method of claim 56, wherein the reporter protein is a luciferase or a fluorescent protein.

58. The method of claim 56, wherein the regulatory region is obtained from a gene that is expressed when the cell exhibits a phenotype of interest.

59. The method of claim 1, wherein the altered phenotype generates an output that comprises production of a detectable signal.

60. The method of claim 59, wherein the signal is electromagnetic radiation.

61. The method of claim 60, wherein the output comprises a pattern of radiation emitted by cells at a plurality of loci.

62. The method of claim 61, wherein the pattern is detected with a charge-coupled device (CCD).

63. The method of claim 1, wherein the phenotype is selected from the group consisting of cell death, alteration in proliferation extent or rate, anchorage dependent growth, a change in trafficking into or within the cell.

64. The method of claim 1, wherein the phenotype is an output that evidences cell proliferation, cell differentiation or protein trafficking.

65. The method of claim 1, wherein the cells are exposed to an effector molecule before, after, or simultaneously with the introduction of the nucleic acid molecule.

66. The method of claim 26, wherein the reporter cells comprise a regulatory region operatively linked to a nucleic acid encoding a reporter protein.

67. The method of claim 66, wherein the reporter protein is a luciferase or a fluorescent protein.

68. The method of claim 66, wherein the regulatory region is obtained from a gene that is expressed when the cell exhibits a phenotype of interest.

69. The method of claim 26, wherein the altered phenotype generates an output that comprises production of a detectable signal.

70. The method of claim 69, wherein the signal is electromagnetic radiation.

71. The method of claim 70, wherein the output comprises a pattern of radiation emitted by cells at a plurality of loci.

72. The method of claim 71, wherein the pattern is detected with a charge-coupled device (CCD).

73. The method of claim 26, wherein the phenotype is selected from the group consisting of cell death, alteration in proliferation extent or rate, anchorage dependent growth, a change in trafficking into or within the cell.

74. The method of claim 26, wherein in the phenotype is an output that evidences cell proliferation, cell differentiation or protein trafficking.

75. The method of claim 1, wherein the cells are exposed to a small effector molecule before, after with the introduced nucleic acid molecule.

76. The method of claim 26, wherein the perturbagen comprises a compound or condition that is an antagonist of a expression of a gene.

77. The method of claim 76, wherein prior to exposure to the antagonist, the cells are exposed to an agonist of expression of the gene.

78. The method of claim 26, wherein the perturbagen is a compound that is an agonist of expression of a gene.

79. The method of claim 1, wherein the cells are exposed to a change in an extracellular condition.

80. The method of claim 26, wherein the cells are exposed to a change in an extracellular condition.

81. The method of claim 79, wherein the change in condition comprises a change in pH, ionic strength, temperature or oxygen content of the external medium.

82. The method of claim 80, wherein the change in condition comprises a change in pH, ionic strength, temperature or oxygen content of the external medium.

83. The method of claim 1, wherein the addressable collection comprises an array.

84. The method of claim 1, wherein the nucleic acid that is introduced comprises a cDNA library, wherein a different member or permutation of members of the library is introduced at each address.

85. The method of claim 1, wherein the nucleic acid that is introduced comprises a library of siRNA, wherein a different member or permutation of members of the library is introduced at each address.

86. The method of claim 1, wherein the introduced nucleic acid molecules are provided as an array and the collection of cells and the array of nucleic acid molecules are contacted under conditions whereby the nucleic acid is introduced into the cells.

87. The method of claim 86, wherein the nucleic acids are linked to discrete loci on a solid support and the cells are added to each locus.

88. The method of claim 87, wherein the loci comprise wells.

89. The method of claim 1, wherein the collection of cells comprises a control cell.

90. The method of claim 89, wherein the reporter cell comprises a reporter construct and the control cell is a cell that is substantially identical to a reporter cell except that it does not comprise a reporter construct.

91. The method of claim 89, wherein the control is a cell that is substantially identical to the other cells in the collection except that nucleic acid is not introduced at step a).

92. The method of claim 89, wherein the control cell comprises a different introduced nucleic acid from the cells that exhibit a change in phenotype.

93. The method of claim 26, wherein the nucleic acid molecules are introduced prior to exposing them to a perturbagen.

94. The method of claim 26, wherein the nucleic acid molecules are introduced after exposing them to a perturbagen.

95. The method of claim 26, wherein the nucleic acid that is introduced comprises a cDNA library, wherein a different member or permutation of members of the library is introduced at each address.

96. The method of claim 26, wherein the nucleic acid that is introduced comprises a library of siRNA, wherein a different member or permutation of members of the library is introduced at each address.

97. The method of claim 26, wherein the addressable collection comprises an array.

98. The method of claim 97, wherein the cells are arrayed in a multi-well plate.

99. The method of claim 98, wherein the plate comprises at least 300 wells.

100. The method of claim 99, wherein the plate comprises at least 1500 wells.

101. The method of claim 26, wherein the introduced nucleic acid molecules are provided as an array and the collection of cells is contacted with the array of nucleic acid molecules under conditions whereby the nucleic acid is introduced into the cells.

102. The method of claim 101, wherein the nucleic acids are linked to discrete loci on a solid support and the cells are added to each locus.

103. The method of claim 102, wherein the loci comprise wells.

104. The method of claim 26, wherein the collection of cells comprises a control cell.

105. The method of claim 104, wherein the reporter cell comprises a reporter construct and the control is a cell that is substantially identical to a reporter cell except that it does not comprise a reporter construct.

106. The method of claim 104, wherein the control is a cell that is substantially identical to the other cells in the collection except that nucleic acid is not introduced at step b).

107. A method of identifying cDNA that, when expressed in a cell, causes an altered response of the cell to a biologically active molecule compared to a control cell, the method comprising:
(a) providing a plurality of reporter cells that each comprises the cell and a construct that comprises nucleic acid encoding a product operably linked to a promoter such that the cDNA is expressed in the reporter cell, wherein different nucleic acid molecules are expressed in each of the plurality of reporter cells;
(b) contacting the each of plurality of reporter cells with a biologically active molecule or expositing the cells to a condition that alters gene expression; and
(c) identifying any reporter cells that have an altered response to the biologically active molecule or the condition compared to a control.

108. A database produced by the method of claim 15.

109. A database produced by the method of claim 41.

110. A combination, comprising:
a) an addressable collection of reporter cells, wherein: the reporter cells generate an output representative of expression of a gene or a cellular activity; and the reporter cells comprise a promoter operatively linked to a reporter gene; and
b) a library of nucleic acid molecules.

111. The combination of claim 110, wherein the promoter is obtained from a gene that is expressed when the cell exhibits a phenotype of interest.

112. The combination of claim 110, wherein the cells are present as populations of cells and the cells of a first population of cells comprise a different member of the library of nucleic acid molecules than cells of at least a second population of cells.

113. The combination of claim 112, wherein each of the cell populations is not in fluid contact with other cell populations.

114. The combination of claim 113, wherein each cell population of the addressable collection is in a well of micro-well plate.

115. The combination of claim 114, wherein the micro-well plate comprises 384 or 1536 wells.

116. The combination of claim 110, wherein the library comprises a library of siRNA.

117. A kit comprising the combination of claim 110; and optionally comprising any additional components selected from the group consisting of instructions for use of the kit for identifying targets of perturbations of gene expression or cellular activity, reagents for introducing the nucleic acid molecules into the cells.

118. A method for identifying the target of an effector or a target for an effector of gene expression or for a cellular activity, comprising:
a) providing an addressable collection of reporter cells, wherein the reporter cells generate an output representative of expression of the gene or the cellular activity;
b) contacting the cells with an effector of the activity or expression;
c) introducing nucleic acid encoding a potential target of the effector, wherein the contacting and introducing step are performed either simultaneously or sequentially in either order; and
d) identifying cells in the collection that exhibit expression or activity that is different in the presence of the nucleic acid than in its absence, thereby identifying the target of or for an effector of gene expression or a cellular activity.

119. The method of claim 118, wherein the collection of cells is provided in a positionally addressable array.

120. The method of claim 118, wherein the collection of cells is provided as populations of cells, each of which populations comprises a different introduced nucleic acid and is not in fluid contact with other cell populations.

121. The method of claim 40, further comprising, contacting the collection of cells with an uncharacterized perturbagen; and comparing the results to recorded data obtained using a characterized perturbagen to identify the class of perturbagen or identity of the perturbagen.

122. The method of claim 1, wherein the introduced nucleic acids encode a product of the endogenous gene.


SEQUENCE LISTING (14 sequences)

SEQ ID NO: 1 - DNA, 32 bp, Artificial Sequence
  primer
ggcctagtcctcgaggggaatttccgggaatt

SEQ ID NO: 2 - DNA, 31 bp, Artificial Sequence
  primer
ggcctagtcggatccttacacggcgatcttt

SEQ ID NO: 3 - DNA, 4987 bp, Artificial Sequence
  pNF_B-Luc vector (Clontech)
ggtaccgagctcttacgcgtgctagcgggaatttccgggaatttccgggaatttccgggaatttccagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 4 - DNA, 34 bp, Artificial Sequence
  Lox P site
ataacttcgtataatgtatgctatacgaagttat

SEQ ID NO: 5 - DNA, 1032 bp, Escherichia coli
  CDS (1)...(1032): nucleotide sequence encoding Cre recombinase
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 6 - protein, 343 aa, Escherichia coli
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 7 - DNA, 1272 bp, Saccharomyces cerevisiae
  CDS (1)...(1272): nucleotide sequence encoding Flip recombinase
atgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 8 - protein, 422 aa, Saccharomyces cerevisiae
PQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 9 - DNA, 66 bp, Bacteriophage mu
  CDS (1)...(66): nucleotide sequence encoding GIN recombinase
tcaactctgtataaaaaacaccccgcgaaacgagcgcatatagaaaacgacgatcgaatcaattaa

SEQ ID NO: 10 - protein, 21 aa, bacteriophage mu
STLYKKHPAKRAHIENDDRIN

SEQ ID NO: 11 - DNA, 69 bp, Bacteriophage mu
  CDS (1)...(69): nucleotide sequence encoding Gin recombinase
tataaaaaacatcccgcgaaacgaacgcatatagaaaacgacgatcgaatcaatcaaatcgatcggtaa

SEQ ID NO: 12 - protein, 22 aa, bacteriophage mu
  Gin recombinase of bacteriophage mu
YKKHPAKRTHIENDDRINQIDR

SEQ ID NO: 13 - DNA, 555 bp, Escherichia coli
  CDS (1)...(555): nucleotide sequence encoding PIN recombinase
atgcttattggctatgtacgcgtatcaacaaatgaccagaacacagatctacaacgtaatgcgctgaactgtgcaggatgcgagctgatttttgaagacaagataagcggcacaaagtccgaaaggccgggactgaaaaaactgctcaggacattatcggcaggtgacactctggttgtctggaagctggatcggctggggcgtagtatgcggcatcttgtcgtgctggtggaggagttgcgcgaacgaggcatcaactttcgtagtctgacggattcaattgataccagcacaccaatgggacgctttttctttcatgtgatgggtgccctggctgaaatggagcgtgaactgattgttgaacgaacaaaagctggactggaaactgctcgtgcacagggacgaattggtggacgtcgtcccaaacttacaccagaacaatgggcacaagctggacgattaattgcagcaggaactcctcgccagaaggtggcgattatctatgatgttggtgtgtcaactttgtataagaggtttcctgcaggggataaataa

SEQ ID NO: 14 - protein, 184 aa, Escherichia coli [sequence omitted]